(19) United States
(12) Patent Application Publication
(10) Pub. No.: US 2023/0241195 A1
(43) Pub. Date: Aug. 3, 2023

(54) MICROENCAPSULATED ORAL STERNE VACCINE

(71) Applicant: THE TEXAS A&M UNIVERSITY SYSTEM, College Station, TX (US)

(72) Inventors: Jamie B. Felix, Bryan, TX (US); Sankar P. Chaki, Bryan, TX (US); Walter E. Cook, Milano, TX (US); Allison Ficht, College Station, TX (US); Thomas A. Ficht, College Station, TX (US)

(21) Appl. No.: 18/009,095

(22) PCT Filed: Jun. 10, 2021
(86) PCT No.: PCT/US2021/036834

§ 371 Date: Dec. 8, 2022

Related U.S. Application Data
(60) Provisional application No. 63/037,330, filed on Jun. 10, 2020.

(51) Int. Cl. A61K 39/07; A61K 9/00; A61K 9/50; A61K 9/48

(52) U.S. Cl. CPC A61K 39/07; A61K 9/0053; A61K 9/5052; A61K 9/4866; A61K 2039/52

(57) ABSTRACT

Methods and compositions for the immunization of animals and humans using an oral immunization or vaccine that comprises B. anthracis Sterne strain 34F2 spores suspended in alginate and coated with a shell containing poly-L-lysine (PEL), a vitelline protein B (VpB), or both and an external coating of alginate in an amount sufficient to protect an animal or human from a lethal dose of anthrax.

### CROSS-REFERENCE TO RELATED APPLICATIONS

This application claims priority to U.S. Provisional Application Ser. No. 63/037,330, filed Jun. 10, 2020, the entire contents of which are incorporated herein by reference.

### TECHNICAL FIELD OF THE INVENTION

The present invention relates in general to methods and compositions for the treatment of Bacillus sp.-induced diseases, and more particularly, to a method of making a novel oral vaccine against Bacillus anthracis.

### STATEMENT OF FEDERALLY FUNDED RESEARCH

Not applicable.

### REFERENCE TO A SEQUENCE LISTING

Not applicable.

### BACKGROUND OF THE INVENTION

Without limiting the scope of the invention, its background is described in connection with vaccines.

Anthrax infections have plagued humans and animals alike for millennia, possibly even causing the fifth and sixth plagues of Egypt.¹ The causative agent, Bacillus anthracis, has been studied since the beginning of microbiology but even after more than a century of scientific studies, the anthrax vaccination field has made little progress, especially a veterinary anthrax vaccine.^{1,2} Consolidated data from the last twenty years found a worldwide distribution with reports of the disease on every habitable continent, yet most animals remain unvaccinated.³ While it may be prudent to mention that the incidence of human infection can be decreased with adequate livestock and wildlife vaccination policies, it should also be of great concern that free-ranging livestock and wildlife populations worldwide are unprotected against anthrax outbreaks that can cause catastrophic harm to sensitive wildlife conservation efforts.³⁻⁶

The current veterinary vaccine, historically referred to as the Sterne vaccine, uses B. anthracis Sterne strain 34F2 spores (Sterne spores) that have naturally lost the pXO2 plasmid and therefore can no longer produce the poly-γ-D-glutamic acid capsule, also known as the anti-phagocytic capsule.⁶ The original formulation of the Sterne vaccine, which is still in use today, consists of Sterne spores suspended in saponin and has been used to vaccinate domesticated livestock against anthrax since its discovery in the late 1930's.^{1,7} Despite decades of successful protections, the Sterne vaccine is outdated, impractical, known to vary in its potency and can cause adverse reactions, occasionally even death.⁸ The Sterne vaccine is administered as a subcutaneous injection, which is a highly impractical method of vaccination for free-ranging livestock and wildlife.¹ Without a reasonable method of wildlife vaccination, yearly anthrax outbreaks in national parks and other wildlife areas worldwide pose economic, ecological and conservational burdens to wildlife health professionals.^{3,7,9,10} Even with these yearly outbreaks, the anthrax spore distribution in these areas is undetermined so it isn't possible to vaccinate wildlife based on an estimated risk of exposure.¹¹ The most feasible way to protect wildlife in these areas would be via oral vaccination however, after results from a previous study demonstrated that the Sterne vaccine is incapable of eliciting an immune response following oral vaccination, the urgent need for an effective oral anthrax vaccine for wildlife has never been more evident.¹²

Other research groups in the oral anthrax vaccination field have reported encouraging results from vaccines expressing a recombinant form of anthrax protective antigen in a variety of bacterial, viral or plant-based expression systems.¹³⁻¹⁶ Unfortunately, exposure to a single recombinant antigen may not stimulate sufficient immune activity to protect against fully virulent exposure. Studies have demonstrated that immunizing mice and guinea pigs with inactivated anthrax spores and recombinant antigens elicited enhanced protection against B. anthracis suggesting that anthrax spore associated antigens are also important for vaccine induced protection.^{17,18} However, inactivated spores and recombinant antigens remain less protective than live-attenuated vaccines.¹⁷

Despite these improvements, what is needed is an improved immunization that protects against anthrax outbreaks that can cause catastrophic harm to sensitive wildlife conservation efforts.

### SUMMARY OF THE INVENTION

The present invention relates to methods and compositions for the treatment of Bacillus-induced diseases and disorders. In preferred embodiments, the invention relates to vaccines.

In one embodiment, the present invention includes an oral immunization against Bacillus anthracis comprising: B. anthracis Sterne strain 34F2 spores suspended in alginate and coated with a shell containing poly-L-lysine (PLL), vitelline protein B (VpB), or both in an amount sufficient to protect an animal or human from a lethal dose of anthrax. In one aspect, the composition further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores. In another aspect, the vitelline protein B is a recombinant protein. In another aspect, the vitelline protein B is encoded by Fasciola hepatica. In another aspect, B. anthracis Sterne strain 34F2 spores encapsulated in alginate and coated with the VpB shell survive exposure to gastric juices. In another aspect, the immunization further comprises a pharmaceutically acceptable carrier, a bait additive, or both. In another aspect, the oral immunization further includes an outer alginate shell surrounding the protein shell that comprises an alginate bead or an alginate microsphere. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM. In another aspect, the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

In another embodiment, the present invention includes a vaccine comprising: B. anthracis Sterne strain 34F2 spores suspended in alginate and coated with a shell containing poly-L-lysine (PLL), a vitelline protein B (VpB), or both, wherein the spores are provided in an amount sufficient to protect an animal or human from a lethal dose of anthrax formulated for oral administration. In one aspect, the composition further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores. In another aspect, the vitelline protein B is a recombinant protein. In another aspect, the vitelline protein B is encoded by Fasciola hepatica. In another aspect, B. anthracis Sterne strain 34F2 spores encapsulated in alginate and coated with the VpB shell survive exposure to gastric condition. In another aspect, the immunization further comprises a pharmaceutically acceptable carrier, a bait additive, or both. In another aspect, the vaccine further comprises an outer shell surrounding the protein shell comprising an alginate bead or an alginate microsphere. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM. In another aspect, the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

In one embodiment, the present invention includes a method for prophylaxis, amelioration of symptoms, or any combinations thereof against Bacillus anthracis in a human or animal subject comprising the steps of: identifying the human or animal subject in need of the prophylaxis, amelioration of symptoms, or any combinations thereof against Bacillus anthracis; and administering a therapeutically effective amount of an attenuated oral immunization against Bacillus anthracis comprising: B. anthracis Sterne strain 34F2 spores suspended in alginate, and the alginate is coated with a shell containing poly-L-lysine (PLL), a vitelline protein B (VpB), or both, wherein the immunization is provided in an amount sufficient to protect an animal or human from a lethal dose of anthrax. In one aspect, the composition further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores. In another aspect, the vitelline protein B is a recombinant protein. In another aspect, the vitelline protein B is encoded by Fasciola hepatica. In another aspect, the B. anthracis Sterne strain 34F2 spores encapsulated in alginate and coated with the VpB shell survive exposure to gastric conditions. In another aspect, the immunization further comprises a pharmaceutically acceptable carrier, a bait additive, or both. In another aspect, the method further comprises an outer shell surrounding the protein shell comprising an alginate bead or an alginate microsphere. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM. In another aspect, the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

In one embodiment, the present invention includes a method of making an attenuated oral vaccine against anthrax (Bacillus anthracis) comprising: suspending a B. anthracis Sterne strain 34F2 spores in alginate, and coating the alginate with a protein shell comprising: poly-L-lysine (PLL), vitelline protein B (VpB), or both, wherein the protein shell protects the spores from exposure to gastric conditions, wherein the amount of the vaccine is sufficient to protect an animal or human from a lethal dose of anthrax. In one aspect, the composition further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores. In another aspect, the vitelline protein B is a recombinant protein. In another aspect, the vitelline protein B is from Fasciola hepatica. In another aspect, the immunization further comprises a pharmaceutically acceptable carrier, a bait, or both. In another aspect, the method, further comprises encapsulating the spores in an alginate bead or an alginate microsphere. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM. In another aspect, the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

### BRIEF DESCRIPTION OF THE DRAWINGS

For a more complete understanding of the features and advantages of the present invention, reference is now made to the detailed description of the invention along with the accompanying figures and in which:

FIG. 1 shows Sterne spore titer response to simulated gastrointestinal environments. Simulated gastric (0.2% (w/v) NaCl, pH 2 and pH 5) and intestinal (0.68% (w/v) KH₂PO₄, pH 7 and 8) fluids were inoculated with 6.8×10⁵ Bacillus anthracis Sterne strain 34F2 spores and incubated overnight at 37° C. with shaking. MOPS buffer (10 mM MOPS, 0.85% NaCl) was also inoculated with 6.8×10⁵ Sterne spores to serve as a negative control for encapsulated vaccine storage conditions. The resulting viable bacterial titer in each solution was determined by plating serial dilutions. Differences between starting and resulting titers were determined by Student's t-tests with **, p<0.01.

FIG. 2 is a graph that shows microcapsule diameter changes to simulated gastrointestinal environments. Microcapsules with and without the protein shell were suspended in simulated gastric (0.2% (w/v) NaCl, pH 2 and pH 5) and intestinal (0.68% (w/v) KH₂PO₄, pH 7 and 8) fluids for 30 and 90 minutes at 37° C. with shaking. Microcapsule samples were also suspended in MOPS buffer (10 mM MOPS, 0.85% NaCl) as a negative control for encapsulated vaccine storage conditions. The capsule diameters after exposure to simulated gastrointestinal fluids were observed in brightfield and measured in ImageJ. Data is reported as the average capsule diameter for the group in μm±the standard deviation. Data for microcapsules without the protein shell is not shown. Significant differences from pre-exposure diameters in MOPS within the same group are identified as ***, p<0.001. Differences between exposure diameters in microcapsules with and without the protein shell after the 30 minute incubation are identified with c, p<0.001 and d, p<0.0001. Differences between exposure diameters in microcapsules with and without the protein shell after the 90 minute incubation are identified with w, p<0.05 and z, p<0.0001.

FIG. 3 shows microcapsule response with the protein shell to simulated gastrointestinal environments. Representative brightfield images of microcapsule samples following exposure to simulated gastric (0.2% (w/v) NaCl, pH 2 and pH 5) and intestinal (0.68% (w/v) KH₂PO₄, pH 7 and 8) fluids for 30 and 90 minutes at 37° C. with shaking. Microcapsule samples were also suspended in MOPS buffer (10 mM MOPS, 0.85% NaCl) as a negative control for encapsulated vaccine storage conditions.

FIGS. 4A and 4B show Sterne spore entrapment in microcapsules. FIG. 4A—Visual and pixel intensity comparison between Low and High Dose Capsules demonstrates the difference (p<0.0001) between the encapsulated doses. FIG. 4B—A considerable amount of aggregated Sterne cells are still entrapped within the microcapsules, as seen in this close up image of a single High Dose microcapsule 56 days after starting the in vitro release experiment (left). Magnified images of vegetative cells (top right) and spores (bottom right) that remain entrapped within the High Dose microcapsule.

FIG. 5 is a graph that shows in vitro release from microcapsules with the protein shell. A 1 ml sample of microcapsules with the protein shell was suspended in 10 ml MOPS at 37° C. with shaking. The MOPS buffer was completely removed and replaced each day. The collected supernatant was serially diluted and plated onto LB agar to quantify the CFU that had been released each day.

FIGS. 6A and 6B are IgG responses from subcutaneous, FIG. 6A and oral vaccination FIG. 6B with Empty Capsules, Sterne Vaccine, Low Dose Capsules and High Dose Capsules. BALBc/J mice were either subcutaneously injected (FIG. 6A) or orally inoculated (FIG. 6B) with 10⁶ unencapsulated B. anthracis Sterne strain 34F2 spores or 10⁶ encapsulated Sterne spores in Low Dose Capsules. An additional group of mice were subcutaneously injected with 10⁹ encapsulated Sterne spores in High Dose Capsules (FIG. 6A). All capsule vaccines were coated with the protein shell. The control groups received Empty Capsules. Serum samples were collected at 0, 15, 31, 43- and 55-days post vaccination and analyzed by ELISA. Antibody responses were analyzed by one-way ANOVA followed by the Tukey-Kramer HSD test and are shown as mean absorbances at 450 nm±standard deviation from the 1:2,000 dilution for subcutaneously inoculated mice and from the 1:125 dilution for orally inoculated mice. Significant differences from pre-vaccination (Day 0) within the same group are identified as ***, p<0.0001. Differences between responses to the Sterne Vaccine and Low Dose Capsules at corresponding time points are identified with a, p<0.05; c, p<0.001 and d, p<0.0001. Differences between responses to the Low Dose Capsules and High Dose Capsules at corresponding time points are identified with x, p<0.01 and y, p<0.0001.

FIG. 7 shows in vitro toxin neutralizing abilities of antibodies from subcutaneous and orally administered Sterne Vaccine, Low Dose Capsules and High Dose Capsules. Serum was collected from mice at 0, 15, 31, 43- and 55-days post subcutaneous or oral vaccination with 10⁶ unencapsulated B. anthracis Sterne strain 34F2 spores, 10⁶ encapsulated Sterne spores in Low Dose Capsules or 10⁹ encapsulated Sterne spores in High Dose Capsules. Both Low and High Dose Capsules vaccines were coated with the protein shell. Control groups received empty capsules (results not included in this graph). Diluted serum samples were pre-incubated with LeTx then added to J774A.1 cells and the resulting cell viability was assessed with MTT dye. Data presented here represents the average absorbance at 595 nm+standard deviations for each group at each time point at a 1:50 dilution. Significant differences from pre-vaccination (Day 0) within the same group are identified as ***, p<0.0001. Differences between the Sterne Vaccine and Low Dose Capsules at corresponding time points are identified with b, p<0.01 and d, p<0.0001. Differences between subcutaneous and oral vaccination responses with the same vaccines at corresponding time points are identified with m, p<0.0001. Differences between responses to the Low Dose Capsules and High Dose Capsules at corresponding time points were not significant.

FIGS. 8A to 8C are illustrations of microcapsules used in this study. (FIG. 8A) Empty Capsules coated with the protein shell, the protein shell can be poly-L-lysine (PLL), vitelline protein B, or both. (FIG. 8B) Low Dose Capsules loaded with Sterne spores and coated with protein shell, again the protein shell can be PLL, vitelline protein B, or both. High Dose Capsules (not pictured) were also prepared like the Low Dose Capsules but with a higher amount of Sterne spores. (FIG. 8C) PLL Capsules loaded with Sterne spores and coated only with PLL. Created with BioRender.com.

FIG. 9 is a graph that shows microcapsule diameter changes for different microcapsule formulations to simulated gastrointestinal environments. Microcapsules coated with just poly-L-lysine, and microcapsules coated with poly-L-lysine and VpB were suspended in simulated gastric (0.2% (w/v) NaCl, pH 2 and pH 5) and intestinal (0.68% (w/v) KH₂PO₄, pH 7 and 8) fluids for 30 and 90 minutes at 37° C. with shaking. Microcapsule samples were also suspended in MOPS buffer (10 mM MOPS, 0.85% NaCl) as a negative control for encapsulated vaccine storage conditions. The capsule diameters after exposure to simulated gastrointestinal fluids were observed in brightfield and measured in ImageJ. Data is reported as the average capsule diameter for the group in μm.

FIG. 10 is a graph that shows IgG responses in white-tailed deer from subcutaneous (A) and oral vaccination (B) with PLL and VpB Capsules or PLL Capsules. White-tailed deer were either subcutaneously injected the commercial Sterne Vaccine containing 10⁶ unencapsulated B. anthracis Sterne strain 34F2 spores in saponin or were orally vaccinated with 10⁹ encapsulated Sterne spores in PLL and VpB Capsules or PLL Capsules. Serum samples were collected at 0, 14, 28, 42, 56, 84, 112 and 137-days post vaccination for the subcutaneous group, and 0, 14, 28 and 42-days for the oral groups. All serum samples were analyzed by ELISA.

FIG. 11 is a graph that shows in vitro toxin neutralizing abilities of antibodies from subcutaneous administered Sterne Vaccine and orally administered PLL Capsules in white-tailed deer. Serum was collected from white-tailed deer at 0, 14, 28, 42, 56, 84, 112 and 137-days post vaccination following subcutaneous vaccination with 10⁶ unencapsulated B. anthracis Sterne strain 34F2 spores, and 0, 14, 28 and 42-days post vaccination following oral vaccination with 10⁹ encapsulated Sterne spores in PLL Capsules. Diluted serum samples were pre-incubated with LeTx then added to J774A.1 cells and the resulting cell viability was assessed with MTT dye. Data presented here represents the average absorbance at 595 nm.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

An oral vaccine against anthrax (Bacillus anthracis) is urgently needed to prevent annual anthrax outbreaks that are causing catastrophic losses in free-ranging livestock and wildlife worldwide. The Sterne vaccine, the current injectable livestock vaccine, is a suspension of live attenuated B. anthracis Sterne strain 34F2 spores (Sterne spores) in saponin. It is not effective when administered orally and individual subcutaneous injections are not a practical method of vaccination for wildlife.

The present invention is the development of a microencapsulated oral vaccine against anthrax. Evaluating Sterne spore stability at varying pH's in vitro revealed that spore exposure to pH 2 results in spore death, confirming that protection from the gastric environment is of main concern when producing an oral vaccine. Therefore, Sterne spores were encapsulated in alginate and coated with a shell containing poly-L-lysine (PLL) and vitelline protein B (VpB), a non-immunogenic, proteolysis resistant protein isolated from Fasciola hepatica. Capsule exposure to pH 2 demonstrated enhanced acid gel character suggesting that alginate microcapsules provided the necessary protection for spores to survive the gastric environment. Post vaccination IgG levels in BALBc/J mouse serum samples indicated that encapsulated spores induced anti-anthrax specific responses in both the subcutaneous and the oral vaccination groups. Post vaccination IgG levels in white-tailed deer serum samples also indicated that encapsulated spores in PLL capsules induced anti-anthrax specific responses following oral vaccination. Furthermore, the antibody responses from the vaccination routes tested in both species were protective against anthrax lethal toxin in vitro, showing the reliability and convenience of the novel oral vaccine formulation to effectively prevent anthrax in wildlife populations.

Recombinant vitelline protein B (VpB), a non-immunogenic, proteolysis resistant protein, was made as follows. First, ˜25 ng of Fasciola hepatica genomic DNA was isolated and polymerase chain reaction (PCR) was used to amplify the coding region of VpB. Next, the amplified Fasciola hepatica genomic DNA was then cloned into an expression vector to ectopically express VpB in E. coli bacteria. E. coli bacterial stock is stored at −80. Finally, to make the recombinant VpB, the E. coli grown and the recombinant VpB is isolated and purified. The making, isolation and purification of recombinant VpB is taught by the present inventors, in at least, U.S. Patent Publication Nos. 20050260258, 20120156287, and 20170135958, relevant portions incorporated herein by reference.

As used herein, the terms “prevent” and “preventing” include the prevention of the recurrence, spread or onset of a disease or disorder. It is not intended that the present invention be limited to complete prevention. In some embodiments, the onset is delayed, or the severity of the disease or disorder is reduced.

As used herein, the terms “treat” and “treating” are not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

The term “subject” as used herein refers to any mammal, preferably livestock, wildlife, a human patient, or domestic pet. It is intended that the term “subject” encompass both human and non-human mammals, including, but not limited to cervids, bovines, caprines, ovines, equines, porcines, felines, canines, wild-game, such as deer, buffalo, etc., as well as humans. In preferred embodiments, the “subject” is a cervid (e.g., a deer) or a human and it is not intended that the present invention be limited to these groups of animals.

As used herein the term “immunogenically-effective amount” refers to that amount of an immunogen required to generate an immune response (e.g. invoke a cellular response and/or the production of protective levels of antibodies in a host upon vaccination).

In a specific embodiment, the term “pharmaceutically acceptable” means approved by a regulatory agency of the federal or a state government or listed in the U.S. Pharmacopeia or other generally recognized pharmacopeia for use in animals and in humans.

The term “carrier” as used herein refers to a diluent, adjuvant, excipient or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a subject, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

The present invention relates to methods and compositions for the treatment of Bacillus induced diseases and disorders. In preferred embodiments, the invention relates to vaccines. In additional embodiments, the invention relates to formulations capable of releasing said live vaccines at a controlled rate of release in vivo. In further embodiments, the invention relates to an oral B. anthracis Sterne strain 34F2 vaccine.

As previously mentioned, no federally approved or commercially available oral veterinary anthrax vaccines are available anywhere worldwide; there simply are no currently known or published existing vaccine alternatives to prevent annual anthrax outbreaks that are causing catastrophic losses in free-ranging livestock and wildlife worldwide. Additionally, the currently approved human anthrax vaccines in the United States and internationally require intramuscular injections with up to five doses to initiate immunity, followed by yearly injections to prolong immunity.

The present invention provides for controlled release compositions further comprising an attenuated, live B. anthracis Sterne strain 34F2 spore vaccine for oral administration. Drug delivery materials have historically been derived from many sources including commodity plastics and textile industries and have been incorporated into vehicles as diverse as pH responsive hydrogels and polymer microparticles or implants designed for surface or bulk erosion as disclosed in Langer RaP, N. A. (2003) Bioengineering, Food and Natural Products 49, 2990-3006, incorporated herein by reference. In the case of controlled release formulations, a drug is typically released by diffusion, erosion or solvent activation and transport. In most cases, the desired polymer characteristics include biocompatibility, lack of immunogenicity, capability of breakdown by the body and water solubility. Many of the processes used to entrap pharmaceuticals involve harsh organic solvents which are bacteriocidal and capable of denaturing proteins. When considering controlled release vehicles for the entrapment of active enzymes or living cells, new alternatives are needed. A number of milder processes based on established technologies and variations have recently been applied to the delivery of active protein agents such as insulin, erythropoietins and chemokines as provided for in Marschutz et al (2000) Biomaterials 21, 1499-07. Takenaga et al (2002) J Control Release 79, 81-91. and Qiu et al (2003) Biomaterials 24, 11-18, all of which are incorporated by reference, or as encapsulants for living cells to permit transplantation as disclosed in Young et al (2002) Biomaterials 23, 3495-3501, hereby incorporated by reference. The technologies cover a wide range of materials including gelatin-based hydrogels, protein-PEG microparticles, novel PEG copolymers, biodegradable PLGA particles, PLG/PVA microspheres and surface modified nanospheres. Alginate, a naturally occurring biopolymer, is especially well suited to the entrapment of living cells. Alginate is a linear unbranched polysaccharide composed of 1-4′-linked β-D-mannuronic acid and α-L-guluronic acids in varying quantities. Alginate polymers are highly water-soluble and easily crosslinked using divalent cations such as Ca2+ or polycations such as poly-L-lysine as provided for in Wee & Gombotz (1998) Adv Drug Deliv Rev 31, 267-285, hereby incorporated by reference. The relatively mild conditions required to produce either an alginate matrix or particle is compatible with cell viability. Entrapment in alginate has been shown to greatly enhance viability and storage as provided for in Cui et al (2000) Int J Pharm 210, 51-59 and Kwok et al (1989) Proc. Int. Symp. Contol. Release Bioact. Mater. 16, 170-171, both of which are incorporated by reference. The physical properties such as porosity, rate of erosion, and release properties may be modulated through mixing alginates of different guluronic acid composition and through applying different coatings to the matrix as provided for in Wee & Gombotz (1998) Adv Drug Deliv Rev 31, 267-285. While in no way limiting the scope of the present invention, it is generally thought that release of a biomolecule from alginate matrices generally occurs through i) diffusion through pores of the polymer or ii) erosion of the polymer network. In general, the alginate matrix is stabilized under acidic conditions, but erodes slowly at pH of 6.8 or above.

There is strong support for oral vaccination with alginate and alginate/protein encapsulated strains as disclosed in Arenas-Gamboa et al. Infect Immun (2008) vol. 76, 2448-55, Kahl-McDonagh et al (2007) Infect Immun 75, 4923-32, Suckow et al (2002) J Control Release 85, 227-235, Kim et al (2002) J Control Release 85, 191-202, all of which are hereby incorporated by reference. In addition, lyophilization of bacteria in alginate beads extends their viability. Embodiments of the present invention include a storage-stable delivery system that may be administered orally and is generally applicable to a number of select agents.

Pharmaceutical Formulations: The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). In one embodiment, the vaccine is encapsulated using materials described in U.S. Patent Application Publications No. 2005/0260258, 2012/0156287, and 2017/0135958, relevant portions hereby incorporated by reference.

In a preferred embodiment, the active compound and optionally another therapeutic or prophylactic agent are formulated in accordance with routine procedures as pharmaceutical compositions adapted for administration to human beings. Typically, the active compounds for administration are solutions in sterile isotonic aqueous buffer. Where necessary, the compositions can also include a solubilizing agent. Compositions for administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the active compound is to be administered by infusion, it can be dispensed, for example, with an infusion bottle containing sterile pharmaceutical grade water or saline. Where the active compound is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for an oral administration of the active compound. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like. Such vehicles are preferably of pharmaceutical grade.

Further, the effect of the active compound can be delayed or prolonged by proper formulation. For example, a slowly soluble pellet of the active compound can be prepared and incorporated in a tablet or capsule. The technique can be improved by making pellets of several different dissolution rates and filling capsules with a mixture of the pellets. Tablets or capsules can be coated with a film that resists dissolution for a predictable period of time. Even the parenteral preparations can be made long acting, by dissolving or suspending the compound in oily or emulsified vehicles, which allow it to disperse only slowly in the serum.

Compositions for use in accordance with the present invention can be formulated in conventional manner using one or more physiologically acceptable carriers or excipients.

Thus, the compound and optionally another therapeutic or prophylactic agent and their physiologically acceptable salts and solvates can be formulated into pharmaceutical compositions for administration by oral (typically feed/bait or in a liquid) or mucosal (such as buccal or sublingual) administration.

For widespread or herd immunization, the present invention can be provided in bait. The bait can be a generic bait made from, e.g., pellets, hay, grasses, common baiting materials, etc. Generally, the livestock bait will be suitable for use by any species of any age or size, including but not limited to cattle, sheep, goats, horses, mules, donkeys, bison, alpacas, llamas, deer, elk, exotic animals, zoo animals, game animals, and wildlife animals.

For oral administration, the compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. The microencapsulated vaccine gives a controlled release or continual boosting effect. Those formulations with VpB and alginate are described in U.S. Patent Application Publication Nos. 2005/0260258, 2012/0156287, and 2017/0135958, hereby incorporated by reference.

For buccal administration the compositions can take the form of tablets or lozenges formulated in conventional manner.

In addition to the formulations described previously, the compositions can also be formulated as a depot preparation. Such long acting formulations can be administered by implantation (for example subcutaneously or intramuscularly) or by intramuscular injection. Thus, for example, the pharmaceutical compositions can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration. In certain embodiments, the pack or dispenser contains one or more unit dosage forms containing no more than the recommended dosage formulation as determined in the Physician's Desk Reference (62nd ed. 2008, herein incorporated by reference in its entirety).

The active compound and optionally other prophylactic or therapeutic agents can also be administered by infusion or bolus injection and can be administered together with other biologically active agents. Administration can be local or systemic. The active compound and optionally the prophylactic or therapeutic agent and their physiologically acceptable salts and solvates can also be administered by inhalation or insufflation (either through the mouth or the nose). In a preferred embodiment, local or systemic parenteral administration is used.

The present invention uses alginate encapsulation.¹⁹⁻²¹ Alginate is naturally indigestible in mammalian systems which can be implemented as a natural controlled release vehicle.^{22,23} Additionally, the mild gelation conditions permit entrapment of the desired capsule load without significantly affecting the viability.²² Post-gelation, the viability of the capsule load is maintained by stability of the microcapsule, particularly in gastric environments which has proven overwhelmingly beneficial for the development of probiotics.²⁰ Alginate has also demonstrated bio-adhesive properties when interacting with mucosal tissues. Combined with the depot effect of alginate capsules, these bio-adhesive properties ensure that the capsule load is repeatedly released in close proximity to target cells.¹⁹

The novel formulation of the present invention showed the stability of microcapsules as enteric delivery vehicles. The inventors also demonstrated the immunogenicity of microencapsulated Sterne spores and observed a pronounced increase in the resulting antibody response from both subcutaneous and oral vaccination. Moreover, an in vitro toxin challenge revealed that the observed antibody response was protective following oral vaccination showing for the first time that microencapsulated of Sterne spores are an alternative anthrax vaccine formulation capable of efficient and protective vaccination of free-ranging livestock and wildlife.

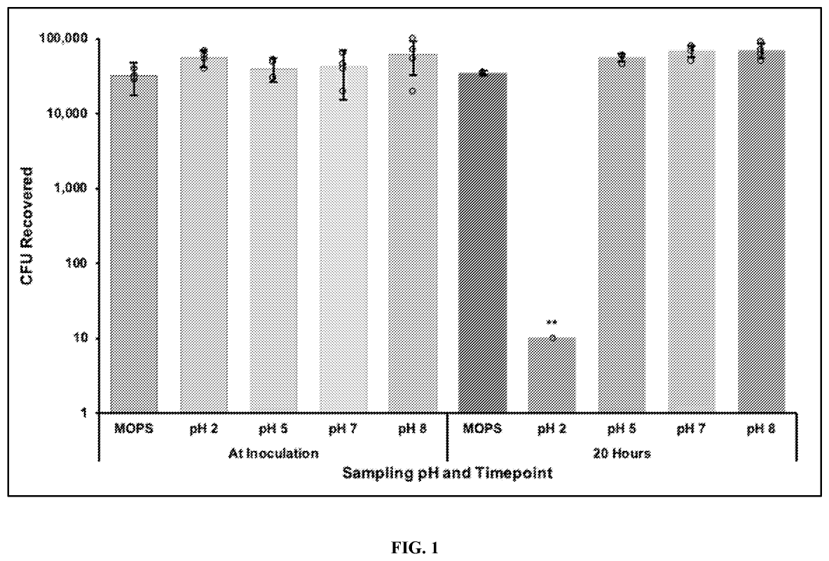

Sterne spore stability in simulated gastrointestinal environments. The unencapsulated Sterne spore response to simulated gastrointestinal fluids (GI fluids) was observed to better understand and account for impairments while in transit through the stomach and intestines. Simulated gastric (0.2% (w/v) NaCl, pH 2 and pH 5) and intestinal (0.68% (w/v) KH₂PO₄, pH 7 and 8) fluids³³ were inoculated with 6.8×10⁵ Bacillus anthracis Sterne strain 34F2 spores and incubated overnight at 37° C. with shaking. MOPS buffer (10 mM MOPS, 0.85% NaCl, [pH 7.4]) was also inoculated with 6.8×10⁵ Sterne spores to serve as a negative control for encapsulated vaccine storage conditions. The unencapsulated Sterne spore titer was severely reduced as a result of exposure to 0.2% NaCl (w/v) pH 2 (p<0.01) with no other significant responses observed from pH 5, 7 or 8 (FIG. 1).

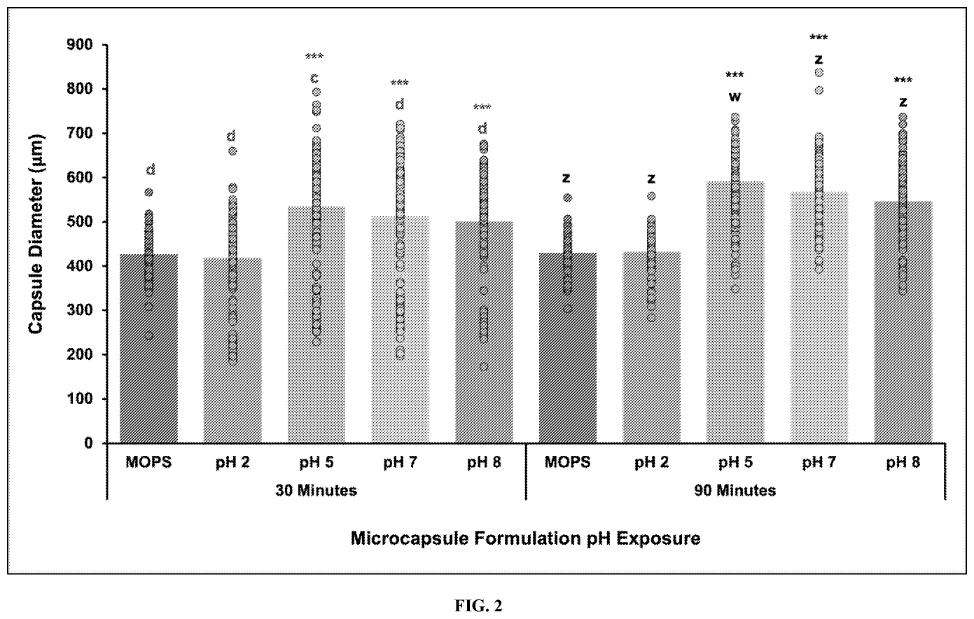

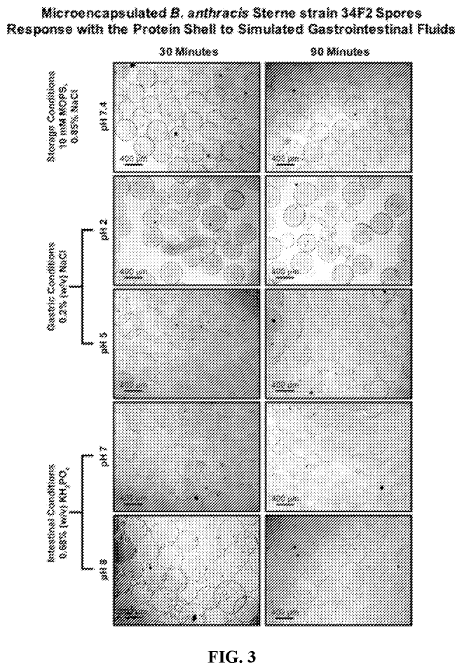

Comparison of microcapsule formulations in gastrointestinal environments. Microcapsules were also exposed to GI fluids to observe the relative stability in simulated gastrointestinal conditions³³ with and without the poly-L-lysine and vitelline protein B shell (protein shell). Microcapsule samples were suspended in MOPS buffer as a negative control and simulated GI fluids at pH 2, 5, 7 and 8 for 30 and 90 minutes at 37° C. with shaking. At pH 2, capsules that were not coated with the protein shell were shown to decrease in diameter compared to neutral storage conditions in MOPS, whereas at pH 5 capsules without the protein shell experienced significant swelling (FIG. 2). The most striking advantage of the protein shell was its capsule stabilization abilities at pH 7 and 8. Without the addition of this proteolysis resistant coating, the capsules completely disintegrated at neutral pHs (FIG. 3). These patterns were also observed in uncoated capsules after 90 minutes in GI fluids, simply to a higher degree as a result of the extended exposure. In comparison, capsules with the protein shell exhibited overall enhanced stability in all GI fluids by preventing shrinking at pH2 and complete capsule dissolution at pH 7 and 8 (FIG. 2).

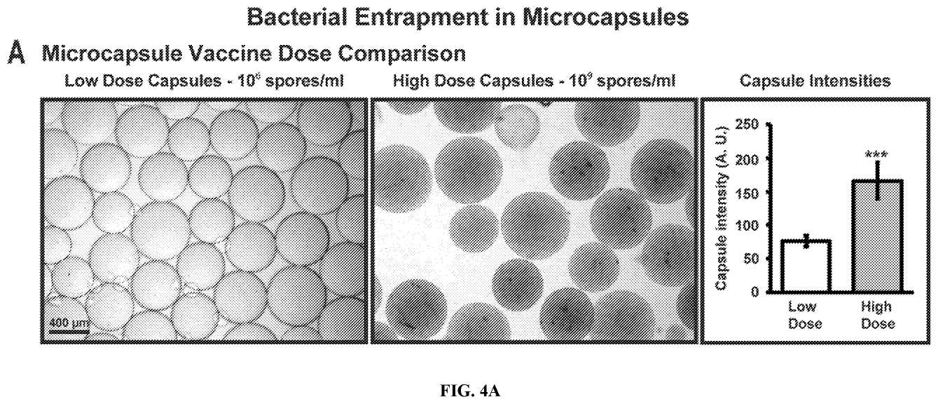

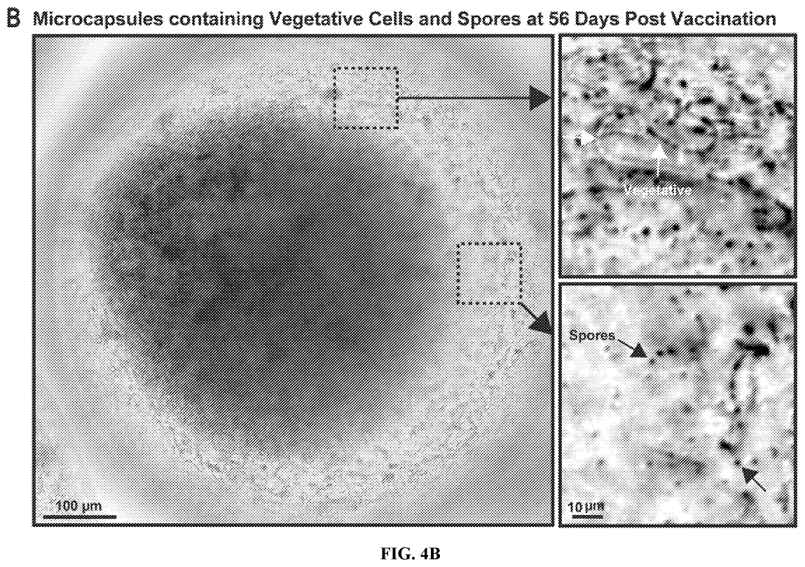

Evidence of bacterial entrapment and controlled release from microcapsules. Microcapsules were imaged in the brightfield to confirm ideal capsule formation and bacterial entrapment. The drastic increase in the amount of encapsulated Sterne spores is visible when comparing the Low Dose Capsules with the High Dose Capsules in storage conditions which were made with 5×10⁶ spores/ml and 4×10¹⁰ spores/ml, respectively (FIG. 4A, left and middle). This significant increase (p<0.0001) is also evidenced by measuring the pixel intensity of the microcapsule images (FIG. 4A, right). An in vitro release experiment was conducted by collecting samples for 38 days to evaluate the timeframe of bacterial release from microcapsules coated with the protein shell. Microcapsules were suspended in 1 ml of MOPS buffer and incubated at 37° C. with shaking. The supernatant was removed and replaced at frequent intervals then serially diluted on LB agar to quantify the release rate. Results depicted in FIG. 5 confirm the sustained release abilities of microcapsules coated with the protein shell. Although the daily sample collection was stopped beyond 38 days, the full experiment was terminated at the same time as the mouse immunization experiment on day 56 when a final bacterial release sample and the remaining capsules were collected analysis (data not shown) and imaging. After 56 days of shaking at 37° C., the capsules still contained aggregations of viable Sterne spores and vegetative cells (FIG. 4B) showing that the capsules continued releasing viable bacteria for much longer.

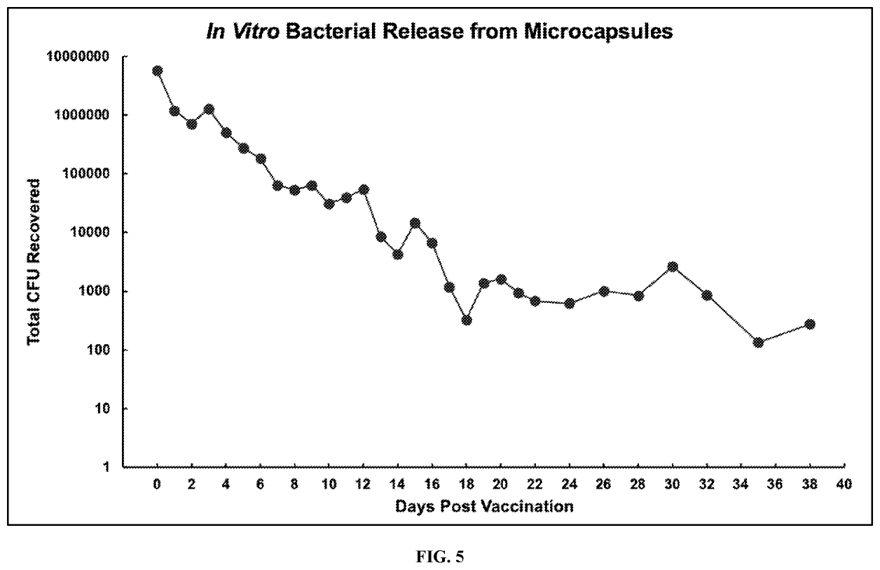

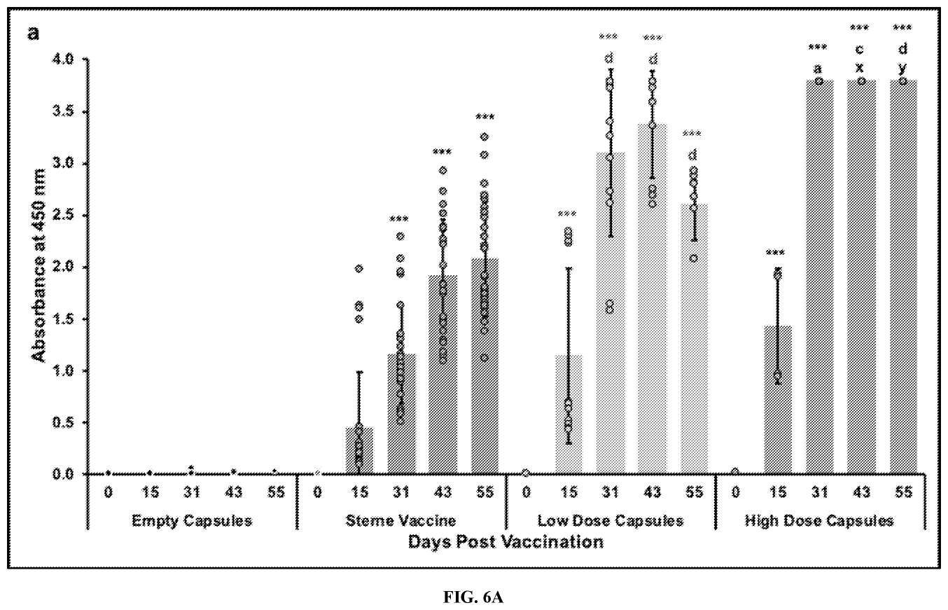

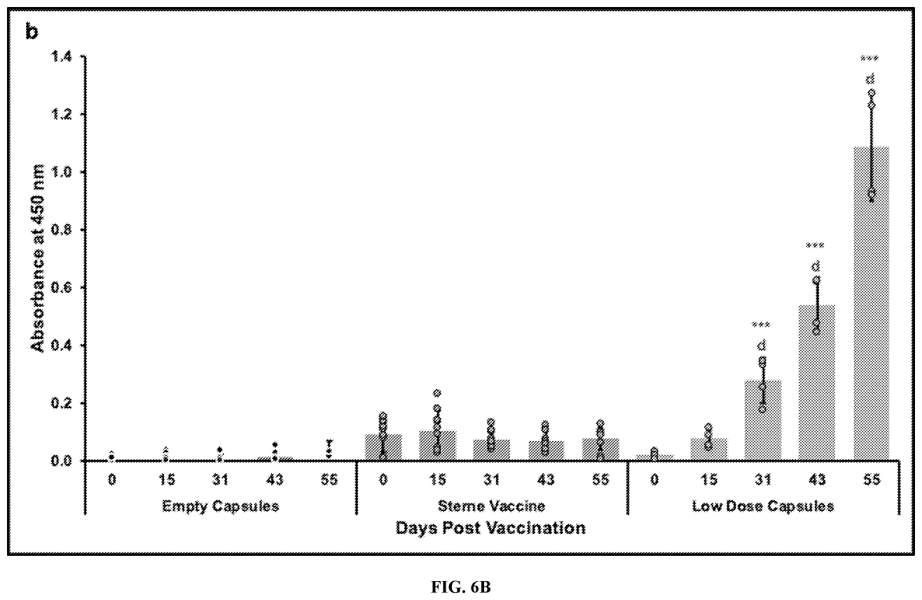

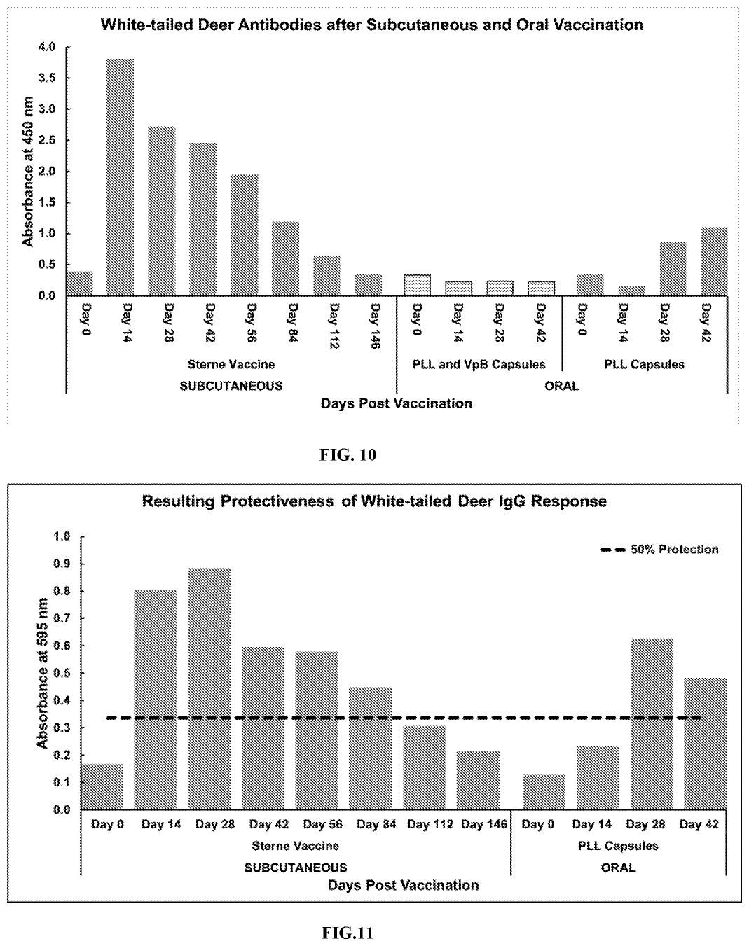

Microcapsule vaccines induce anthrax specific antibody responses. Antibody levels against anthrax protective antigen were measured by ELISA and are illustrated as mean absorbances at 450 nm±the standard deviation in FIGS. 6A,6B and FIG. 10. Antibody titers were also estimated by end-point dilution. All vaccines containing Sterne spores elicited strong antibody responses starting at 15 days post subcutaneous vaccination (FIG. 6A). The Sterne vaccine exhibited a gradual increase with each time point as did the encapsulated vaccine. Despite being inoculated with the same dose of spores, the Low Dose Capsule group demonstrated higher antibody levels than the Sterne vaccine group at all time points past day 15. These antibody levels were even further increased in mice that were subcutaneously vaccinated with the High Dose Capsules. Both the Low and High Dose capsule vaccines displayed extreme antibody level increases at 31 days post vaccination. A similar antibody spike was also observed from the orally administered Low Dose Capsules at 31 days post vaccination and it continued to increase each week like the responses observed from the subcutaneous vaccines (FIG. 6B). Both orally administered vaccines contained the same dose of Sterne spores, but the oral Sterne vaccine did not induce any antibody response. White-tailed deer orally vaccinated with 10⁹ Sterne spores in PLL capsules developed an antibody titer at 28 days post vaccination.

Microencapsulated Sterne spores induce toxin neutralizing antibodies. LeTx neutralization assays evaluated the ability for vaccination induced antibody responses to protect J774A.1 cells from LeTx mediated killing. The toxin neutralizing abilities of all vaccination groups are illustrated in FIG. 7 as mean absorbances at 595 nm+the standard deviation at a single serum dilution of 1:50. Neutralizing antibody titers were estimated with serial dilutions. In agreement with the ELISA results, serum from all subcutaneous vaccines containing Sterne spores were able to prevent LeTx induced mortality in vitro at all measured time points (FIG. 7). The Low Dose Capsule vaccine exhibited enhanced LeTx neutralizing abilities at 31- and 43-days post vaccination with similar improvements induced by the High Dose Capsule vaccine. Strikingly, the oral capsule vaccine also resulted in toxin neutralizing effects at the same dilution as subcutaneously immunized mice. Serum from mice immunized orally with the Sterne vaccine did not provide any protection from LeTx challenge in vitro. When white-tailed deer were immunized orally with PLL capsules, the resulting antibody titers were protective against LeTX induced mortality in vitro starting at 28 days post vaccination (FIG. 11).

The benefits of oral vaccine delivery cannot be overstated, particularly when it comes to protecting free-ranging livestock and wildlife from current and emerging infectious diseases such as anthrax. Development of oral vaccines can allow for easy, wide-spread vaccination policies without needing to deal with the labor-intensive programs and painful injections associated with the majority of today's human and animal vaccines. It is also possible that effective oral vaccines may be intrinsically more stable and have longer shelf-lives as a collateral benefit of the stability required for transit through the gastrointestinal tract. Furthermore, oral vaccines can lead to enhanced efficacy with less adverse effects due to mucosal immunity and oral delivery.

For all of these reasons and more, an alternative anthrax vaccine formulation specifically for oral administration is urgently needed to protect animals worldwide from potentially catastrophic anthrax outbreaks.^{3,12} Many wildlife health professionals have demanded a new veterinary anthrax vaccine because individual hand-injections for each and every animal is not a practical method of vaccination for wildlife and a recent study demonstrated that oral vaccination with the Sterne vaccine is not effective.^{1,12} Also, sustained protection from the Sterne vaccine can only be achieved with yearly boosters which requires a yearly cycle of troublesome injections with the potential for adverse reactions.¹ To resolve the many issues associated with anthrax outbreaks and vaccination, the inventors developed and evaluated a novel anthrax vaccine formulation for oral vaccination. Results of the inventors' study demonstrate that subcutaneous and oral vaccination with microencapsulated B. anthracis Sterne strain 34F2 spores can induce antibody production in the murine model and inactivate B. anthracis lethal toxin in vitro.

Oral vaccination is a common goal throughout the entire vaccinology field but there are still a limited number of oral vaccines approved for animal and human use because the main obstacle facing oral vaccination is, ironically, oral vaccination itself.³⁴⁻³⁶ The principle of oral vaccination is completely dependent on getting sensitive antigens through the harsh, gastric environment that was evolutionarily designed specifically to prevent that exact thing from happening. In contrast, gastrointestinal pathogens, such as anthrax, have also evolved over thousands of years to survive the gastric environment for eventual uptake in the small intestine but these pathogen survival strategies aren't typically conserved in live attenuated organisms, which is a reliable vaccine format. Such is the case with B. anthracis Sterne strain 34F2. Upon exposure to a simulated gastric environment, there was a severe decrease in the viable Sterne spore titer (FIG. 1). This implies that development of an oral vaccine with the Sterne strain must involve some protection to ensure passage through the stomach. Given that the majority of anthrax infections in wildlife are gastrointestinal, it can be reasoned that fully virulent anthrax spores are able to survive passage through a harsh acidic environment to establish infections following uptake in the small intestine. In comparison to the experiments performed here with the pXO2-negative Sterne strain, this suggests that fully virulent anthrax spores may be better equipped to survive the gastrointestinal environment due to retention of the pXO2 plasmid. Alginate encapsulation with the addition of a proteolysis resistant protein shell was able to shield Sterne spores enough through the gastric environment to induce an immune response following oral vaccination.

First, the stabilizing and shielding abilities of the microcapsules produced in this study was assessed by observing the microcapsule responses to simulated gastrointestinal environments. When alginate capsules are formed in a cross-linking solution, guluronate residues in the alginate cooperatively bind Ca²⁺ ions from the solution, thus cross-linking the alginate polymers to the “pre-gel” state.^{21,24} Exposure of a calcium cross-linked pre-gel to nongelling cations, such as Na⁺, will reduce the mechanical stability of the alginate gel and possibly disintegrate the entire polymer matrix, as exhibited in FIG. 3.^{21,25} This can be prevented by adding additional cross-linked layers to the microcapsules, thus resulting in more stable capsules which the inventors have demonstrated here by exposing coated microcapsules to gastrointestinal environments.³⁷

The added stability of these layers can be assessed through changes in microcapsule shrinking, swelling and overall morphology. Changes in the alginate polymer network such as these can greatly affect the rate of diffusion through and the erosion of the network, thereby altering the antigen release rate.^{22,38,39} Results of this study demonstrate the efficacy of using the PLL and VpB protein shell in this microcapsule formulation because the it prevented most of the destabilizing effects of simulated gastrointestinal fluids. Specifically, the protein shell reduced the degree of swelling experienced by the capsules at pH 5, thereby avoiding drastic changes in the polymer network that could have led to premature bacterial release. Of most importance was that the protein shell maintained the capsule integrity at pH 7 and 8, whereas other studies have observed alginate disintegration at pH 7 and 8.^{40,41} By preventing complete capsule dissolution in neutral environments, the protein shell ensures that the capsule is stable enough to serve its controlled release purpose by stimulating mucosal immunity and uptake in the intestines.

A second challenge to oral vaccination, after having endured the harsh gastric environment, is to ensure antigen transport across the intestinal epithelia followed by antigen-presenting cell activation.

Subcutaneous vaccination with Low Dose Capsules enhanced the observed antibody response even though mice received the same dose of spores as those vaccinated with the Sterne vaccine (FIG. 6A). Increasing the encapsulated spore dose also resulted in an even more robust antibody response following subcutaneous vaccination with High Dose Capsules. Excitingly, ELISA results also revealed a significant improvement in the amount of antibody produced following oral vaccination with the Low Dose Capsules when compared to the Sterne vaccine (FIG. 6B), as well as following oral vaccination with PLL Capsules (FIG. 10). To the inventors' knowledge, this is the first time a measurable antibody response has ever been recorded following oral vaccination with live attenuated Sterne spores. The single prior attempt involved mixing Sterne spores with scarifying agents for an oral subcutaneous vaccination by way of tiny lacerations in the gums, tongue, oropharynx, etc. and observed limited success.⁴⁷ In contrast, results presented here were obtained from mice vaccinated by oral gavage which completely bypassed the oral mucosa. Additionally, white-tailed deer were vaccinated by needle free syringe which also mostly bypassed the oral mucosa while still inducing an immune response following oral vaccination with PLL capsules (FIG. 10). This shows that microencapsulation with the protein shell, or other permanent cross-linker like PLL, provides enough protection for Sterne spores to survive the gastric environment and progress into the small intestine to stimulate an immune response.

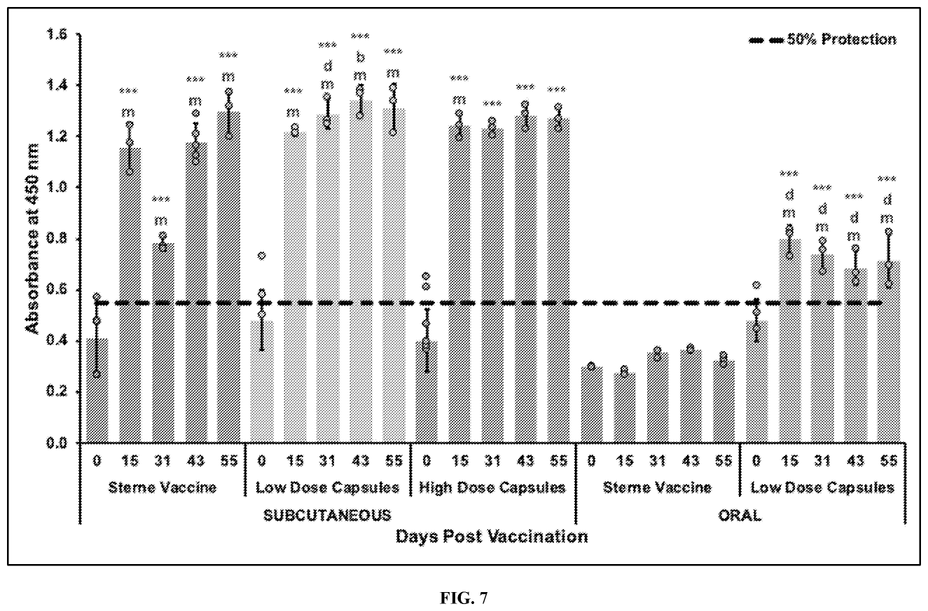

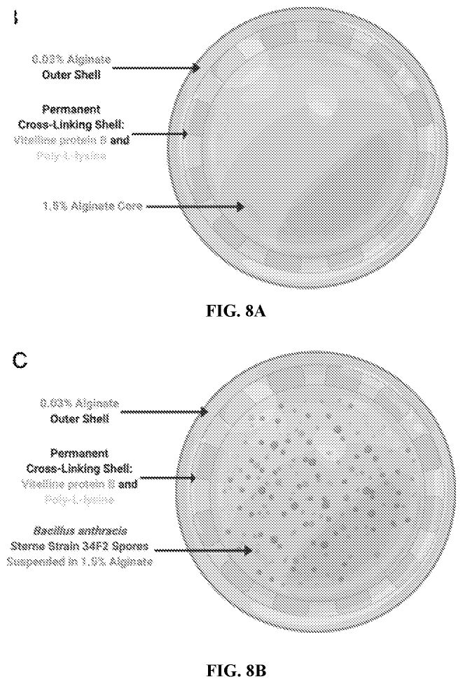

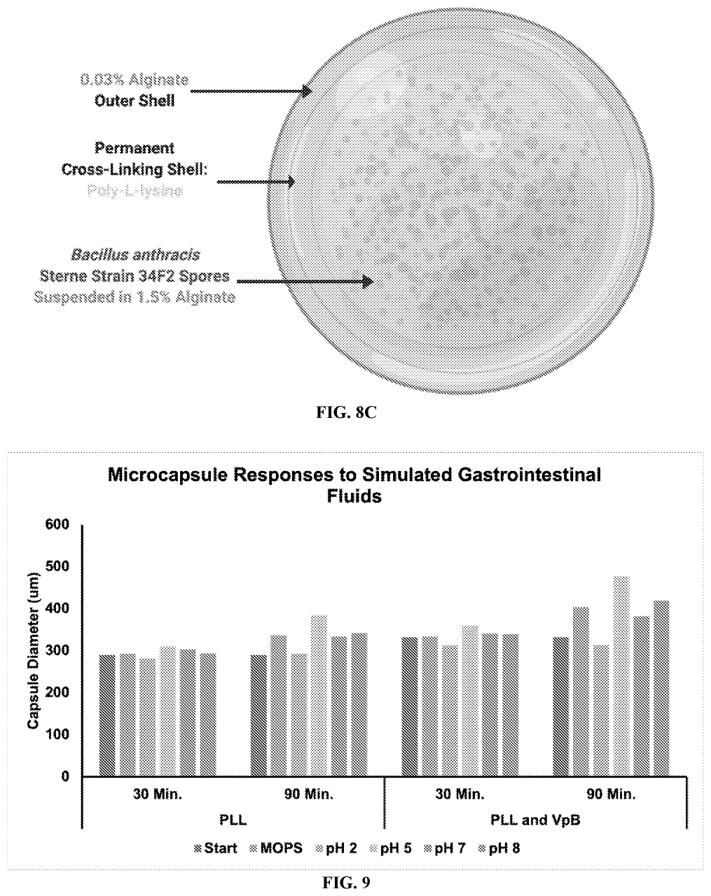

FIGS. 8A to 8C are illustrations of microcapsules used in this study. (FIG. 8A) Empty Capsules coated with the protein shell, the protein shell can be poly-L-lysine (PLL), vitelline protein B, or both. (FIG. 8B) Low Dose Capsules loaded with Sterne spores and coated with protein shell, again the protein shell can be PLL, vitelline protein B, or both. High Dose Capsules (not pictured) were also prepared like the Low Dose Capsules but with a higher amount of Sterne spores. (FIG. 8C) PLL Capsules loaded with Sterne spores and coated only with PLL. Created with BioRender.com.

The advantages of this microcapsule formulation were also detected in results from toxin neutralization assays (FIG. 7, 11) which are considered an additional marker and stronger correlate of protection.^{13-15,48,49} Subcutaneous vaccination with Low Dose Capsules resulted in better protection for cultured macrophages at 31- and 43-days post vaccination when compared to the unencapsulated Sterne vaccine. Additionally, subcutaneous injection with approximately 9×10⁹ Sterne spores per mouse in High Dose Capsules resulted in extraordinarily high serum IgG responses (FIG. 6A) that were fully protective by 15 days post vaccination (FIG. 7). This antibody response also may not yet have reached its peak prior to the end of the experiment. The in vitro release experiment demonstrated that High Dose Capsules were still releasing Sterne spores 38 days after vaccination (FIG. 5) and that there was an excessive amount of Sterne spores and vegetative cells still entrapped within the High Dose Capsules showing that the controlled release could have continued for much longer (FIG. 4B).

According to previous work on mouse susceptibility to B. anthracis strains, the LD₅₀ for BALB/cJ mice subcutaneously injected with the Sterne strain was 6.8×10⁷ spores.⁵⁰ In this study, BALBc/J mice were subcutaneously injected with over 100-fold times more Sterne spores with only one death, implying that this microencapsulation method can allow for enhanced protection with higher Sterne spore doses and less reactogenicity. Inoculation with a higher dose of Sterne spores could also be critical for successful oral vaccination. Sterne spore exposure to acidic environments greatly reduces the viable spore titer (FIG. 1), so vaccinating with a higher dose of microencapsulated Sterne spores may account for any titer loss due to the gastric environment.²⁰

FIG. 9 is a graph that shows microcapsule diameter changes for different microcapsule formulations to simulated gastrointestinal environments. Microcapsules coated with just poly-L-lysine, and microcapsules coated with poly-L-lysine and VpB were suspended in simulated gastric (0.2% (w/v) NaCl, pH 2 and pH 5) and intestinal (0.68% (w/v) KH₂PO₄, pH 7 and 8) fluids for 30 and 90 minutes at 37° C. with shaking. Microcapsule samples were also suspended in MOPS buffer (10 mM MOPS, 0.85% NaCl) as a negative control for encapsulated vaccine storage conditions. The capsule diameters after exposure to simulated gastrointestinal fluids were observed in brightfield and measured in ImageJ. Data is reported as the average capsule diameter for the group in μm.

FIG. 10 is a graph that shows IgG responses in white-tailed deer from subcutaneous (A) and oral vaccination (B) with PLL and VpB Capsules or PLL Capsules. White-tailed deer were either subcutaneously injected the commercial Sterne Vaccine containing 10⁶ unencapsulated B. anthracis Sterne strain 34F2 spores in saponin or were orally vaccinated with 10⁹ encapsulated Sterne spores in PLL and VpB Capsules or PLL Capsules. Serum samples were collected at 0, 14, 28, 42, 56, 84, 112 and 137-days post vaccination for the subcutaneous group, and 0, 14, 28 and 42-days for the oral groups. All serum samples were analyzed by ELISA.

Additional protection observed from the orally administered Low Dose Capsules and PLL capsules. The antibody responses induced by oral vaccination depicted in FIG. 6B were produced from serum diluted 1:125 whereas the subcutaneous antibody responses depicted in FIG. 6A were produced from serum diluted 1:2,000. Despite being much less concentrated according to the ELISA results, the antibody responses induced by oral vaccination with Low Dose Capsules were considered protective against LeTx challenge at the same serum dilution as subcutaneously vaccinated Low Dose Capsules, and even at a higher serum dilution than the subcutaneously vaccinated Sterne Vaccine. Further results were observed in white-tailed deer following vaccination with PLL capsules (FIG. 10).

FIG. 11 is a graph that shows in vitro toxin neutralizing abilities of antibodies from subcutaneous administered Sterne Vaccine and orally administered PLL Capsules in white-tailed deer. Serum was collected from white-tailed deer at 0, 14, 28, 42, 56, 84, 112 and 137-days post vaccination following subcutaneous vaccination with 10⁶ unencapsulated B. anthracis Sterne strain 34F2 spores, and 0, 14, 28 and 42-days post vaccination following oral vaccination with 10⁹ encapsulated Sterne spores in PLL Capsules. Diluted serum samples were pre-incubated with LeTx then added to J774A.1 cells and the resulting cell viability was assessed with MTT dye. Data presented here represents the average absorbance at 595 nm.

Similar to the response from the subcutaneously injected High Dose Capsules, it is also possible that the antibody response due to oral vaccination with Low Dose Capsules had not yet peaked prior to the end of the experiment. In fact, a significant antibody response wasn't even detected until 31 days post vaccination. Given that the gastrointestinal emptying time for a mouse is less than 24 hours,⁵¹ the ELISA data shows that coated capsules containing Sterne spores may be demonstrating the mucoadhesive properties of alginate by adhering to the intestinal lumen to gradually release their bacterial load.^{22,52} This conclusion is also corroborated by the in vitro bacterial release experiment which demonstrated that significant amounts of Sterne spores were still entrapped within High Dose Capsules nearly two months after vaccination (FIG. 4). Continued exposure resulting from extended capsule stability acts as a self-contained booster effect and it is possible that oral vaccination with a higher dose of microencapsulated Sterne spores, or even a booster dose of the same vaccine may further enhance the orally induced immune response.

The findings of this study exemplify the advantages and efficacy of Sterne spore microencapsulation. It is further demonstrated that the protein shell is essential for maintaining the controlled release aspects of alginate microcapsules. The microcapsule formulation of the present invention was also capable of sustaining Sterne spore viability in an acidic environment and of releasing viable Sterne cells for at least 56 days. Following a single vaccination dose in mice, microencapsulated Sterne spores generated a significant antibody response via subcutaneous, but more impressively, oral vaccination, both of which were protective during in vitro LeTx challenge. This immune response can be further enhanced by inoculating a higher bacterial dose with limited adverse effects.

While the results presented here reveal the great potential for this oral vaccine formulation, the majority of wildlife species affected by anthrax are ruminants and thus present further challenges to oral vaccination in the form of three additional stomachs and rumination.⁵³ Continued research is essential to optimize this vaccine for ruminant species. Future work will involve in vivo studies in a ruminant model to evaluate effective oral vaccination doses and the effect of vaccine boosters. It will also be critical to do an in vivo challenge experiment in a ruminant model to fully demonstrate the protective efficacy of this vaccine. The vaccine can be added in a wildlife bait to establish a practical wildlife vaccination method against anthrax.

In summary, the present invention is the first effective oral vaccination against anthrax. It is demonstrated herein, for the first time, the generation of protective antibody responses from oral vaccination with B. anthracis Sterne strain 34F2 spores, which can be adapted such that the Sterne spore is effective for oral vaccination of free-ranging livestock and wildlife.

Preparation of Sterne spores. All bacteria used in this experiment were cultured from a vial of the Anthrax Spore Vaccine (ASV) from Colorado Serum Company (Denver, Colo., USA), the North American commercial producer of the Sterne vaccine. The ASV consists of live attenuated B. anthracis Sterne strain 34F2 spores in saponin which were isolated and cultured as described previously.¹² Briefly, a small volume of Luria Broth (LB) was inoculated with the ASV and cultured overnight at 37° C. with shaking. The growth was pelleted by centrifugation at 3800 rpm for 15 minutes, resuspended in LB broth, then plated onto LB agar and incubated at 37° C. for 6 days to sporulate.⁵⁴⁻⁵⁸ The full bacterial lawns were harvested from the plates and washed repeatedly with sterile water, or 2.5 mM D-alanine or 5 mM D-alanine. The skilled artisan will understand that the amount of D-alanine can be modulated to prevent spore germination, such as 0.1, 0.2, 0.3, 0.4, 0.5. 0.75, 1, 2, 2.5, 3, 4, 5, 6, 7, 8, 9, 10 or more mM. Alternatively, LB was inoculated with the ASV and cultured in the liquid form at 37° C. for 5-7 days, or until sporulated. Spores were harvested by centrifugation and washed repeatedly with sterile water or 2.5 mM D-alanine or 5 mM D-alanine. Remaining vegetative cells were killed by heating at 68° C. for 1 hour and removed by filtering through a 3.1 μm filter, if needed, resulting in a suspension of pure Sterne spores. The final Sterne spore concentration was estimated by plating serial dilutions on LB agar.

Sterne spore response to simulated gastrointestinal environments. B. anthracis Sterne strain 34F2 spores were exposed to simulated gastric or intestinal fluids (GI fluids) to fully comprehend the obstacles to oral vaccination. Simulated gastric fluids consisted of 0.2% (w/v) NaCl and were adjusted to pH 2 and 5 with 1 M HCl to mimic the range of pHs in a non-fasted stomach.³³ Simulated intestinal fluids were 0.68% (w/v) K₂HPO₄ adjusted to pH 7 and 8 with 0.2 M NaOH.³³ The pH range covered by the prepared GI fluids was also representative of the environments throughout the ruminant digestive tract where the pH of the rumen is 6.5-7, the reticulum is ˜6, the omasum is 4-5 and the abomasum is 2-4.^{53,59} A Sterne spore stock solution was prepared at an arbitrary concentration of 3.4×10⁶ spores/ml. From this stock solution, 0.2 ml was used to inoculate 6.8×10⁵ total Sterne spores into 5 ml of each GI fluid and MOPS Buffer (10 mM MOPS, 0.85% NaCl [pH 7.4]) as a control for future vaccine conditions. The starting spore titer of each inoculated GI fluid was determined by plating serial dilutions on LB agar, then the samples were placed on an orbital shaker at 37° C. Sterile water, phosphate buffered saline (PBS) and LB broth were also inoculated as negative and positive controls (data not included here). After an overnight incubation, the resulting spore concentration in the GI fluids was determined by plating serial dilutions. Data are reported as the average total recovered colony forming units (CFU) from each buffer.

Vaccine preparation. Sterne vaccine. The ASV is distributed by Colorado Serum with a recommended 1 ml dose of between 4×10⁶ and 6×10⁶ viable Sterne spores in saponin for use in cattle, sheep, goats, swine and horses.¹² This dosage range was simplified to 5×10⁶ spores/ml for the purposes of this experiment and was used exactly as received from Colorado Serum Company.

Microencapsulation of B. anthracis Sterne strain 34F2 spores. Five different microcapsule vaccine formulations with the PLL and/or VpB coating (protein shell) were prepared for the experiments in this study: (i) microcapsules containing 5×10⁶ spores/ml without the protein shell, (ii) empty microcapsules with the protein shell (Empty Capsules), (iii) microcapsules containing 5×10⁶ spores/ml with the protein shell (Low Dose Capsules); (iv) microcapsules containing 10⁹ spores/ml with only the PLL coating (PLL Capsules), and (v) with the protein shell (High Dose Capsules/PLL and VpB Capsules) (FIGS. 8A, 8B, 8C).

Microcapsules were prepared similar to previous studies.³¹ Sodium alginate (NovaMatrix, Sandvika, Norway) was dissolved in MOPS buffer to a concentration of 1.5% (w/v) alginate. The skilled artisan will understand that the concentration (w/v) of alginate can be selected, such as 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% w/v. To make capsules, Sterne spores were suspended in MOPS buffer, sterile water, 2.5 mM D-alanine or 5 mM D-alanine and then mixed with 5 ml of 1.5% (w/v) alginate solution. Microcapsules were formed using a Nisco Encapsulator VARV1 unit (Nisco Engineering AG, Zurich, Switzerland). The spore+alginate solution was extruded through a 170 μm nozzle, released directly into cross-linking solution (100 mM CaCl₂, 10 mM MOPS) and stirred for 30 minutes. The capsules were thoroughly washed with MOPS and then coated with the protein shell by stirring for 30 minutes in 0.05% PLL and VpB in cross-linking solution. After another washing with MOPS, the capsules received an outer shell of 0.03% (w/v) alginate by mixing for 5 minutes. Final microcapsule vaccines (FIGS. 8A and 8B) were washed and resuspended 1:1 in MOPS for storage at 4° C. until use. Empty Capsules were prepared as above but without any Sterne spores being added to the alginate and High Dose Capsules were prepared with a higher amount of Sterne spores added to the alginate. The resulting dose of viable Sterne spores in the microcapsule vaccine was determined by dissolving 1 ml of capsules in 50 mM sodium citrate, 0.45% NaCl, 10 mM MOPS prior to permanent cross-linking with the protein shell.³¹ All microcapsule batches were visualized in the brightfield and pixel intensities were measured in ImageJ.

Characterization of microcapsules in simulated gastrointestinal environments. Microcapsule morphology and bacterial presence within the alginate capsules were visualized with brightfield microscopy. Capsule responses, with the protein shell to simulated gastrointestinal fluids (GI fluids) were examined by suspending an aliquot of each capsule formulation in separate vials of the GI fluids. Vials were placed on a tube rocker at 37° C. and samples were collected at 30 and 90 minutes for imaging on an Olympus CKX41 microscope. Capsule diameters were measured in ImageJ.

Bacterial release from microcapsules. The bacterial release rates from the microcapsules were examined in vitro by suspending 1 ml of capsules in 9 ml of MOPS buffer and placing the tubes on a rocker at 37° C.³¹ At each sampling time point, the capsules were allowed to settle out of the buffer and then as much of the supernatant as possible was collected without disturbing the capsule pellet. The supernatant was plated on LB agar to estimate the bacterial release since the last time point. Capsules were resuspended in the same volume of MOPS buffer that had been removed and returned to the rocker at 37° C. Samples were collected every day for 22 days, approximately every other day until day 38 and a final sample was collected at day 56 when the mouse study was terminated. Results are reported in terms of bacterial release per time point versus time.

Mouse immunizations. Female BALBc/J mice between four and six weeks of age were purchased from The Jackson Laboratory (Bar Harbor, Me., USA). Upon arrival at the animal facility, mice were randomly distributed into six groups of five mice each (Table 1) and allowed to acclimate for at least a week prior to any manipulation. All animal care and experimental procedures were performed in compliance with the Texas A&M University Institutional Animal Care and Use Committee regulations (AUP #IACUC 2016-0112).

Mice were inoculated subcutaneously or by oral gavage with 0.2 ml of one of the four prepared vaccines: (i) Empty Capsules, (ii) Sterne Vaccine, (iii) Low Dose Capsules and (iv) High Dose Capsules (Table 1). All mice inoculated with either the Sterne vaccine or Low Dose Capsules received approximately 1×10⁶ spores/mouse while mice inoculated with the High Dose Capsules received approximately 9×10⁹ spores/mouse. The Empty Capsules served as the unvaccinated control. Antibody responses were evaluated in blood samples that were collected three to seven days prior to vaccination and then every 10 to 14 days after vaccination for eight weeks.

TABLE 1
Vaccination groups to assess the efficacy of microencapsulated
Sterne spores as an oral vaccine.

| | | | | | Blood Collection |
|---|---|---|---|---|---|
| | | Inoculation | Spores/ | Spores/ | (days post- |
| Route | Group (n = 5) | Volume | ml | Mouse | vaccination) |
| SC | Empty Capsules | 0.2 ml | — | — | 0, 15, 31, 43, 55 |
| | Sterne Vaccine | 0.2 ml | 5 × 10⁶ | 1 × 10⁶ | 0, 15, 31, 43, 55 |
| | Low Dose | 0.2 ml | 5 × 10⁶ | 1 × 10⁶ | 0, 15, 31, 43, 55 |
| | Capsules | | | | |
| | High Dose | 0.2 ml | 4 × 10⁹ | 9 × 10⁹ | 0, 15, 31, 43, 55 |
| | Capsules | | | | |
| Oral | Empty Capsules | 0.2 ml | — | — | 0, 15, 31, 43, 55 |
| | Sterne Vaccine | 0.2 ml | 5 × 10⁶ | 1 × 10⁶ | 0, 15, 31, 43, 55 |
| | Low Dose | 0.2 ml | 5 × 10⁶ | 1 × 10⁶ | 0, 15, 31, 43, 55 |
| | Capsules | | | | |

SC = subcutaneous,
Empty Capsules = Microcapsules with PLL and VpB shell (no bacteria),
Sterne Vaccine = B. anthracis Sterne strain 34F2 spores in saponin,
Low Dose Capsules = Microcapsules with the protein shell and the standard dose of Sterne spores,
High Dose Capsules = Microcapsules with the protein shell and a higher dose of Sterne spores

Deer immunizations. White-tailed deer were inoculated subcutaneously or orally with a (i) a full, 1 ml dose of the commercial Sterne vaccine, (ii) 10⁹ encapsulated Sterne spores in PLL/VpB capsules or (iii) 10⁹ encapsulated Sterne spores in PLL capsules. All animal care and experimental procedures were performed in compliance with the Texas A&M University Institutional Animal Care and Use Committee regulations (AUP #IACUC 2019-0328). Antibody responses were evaluated in blood samples that were collected prior to vaccination, every 10 to 14 days after vaccination for eight weeks, then approximately every 28 days for another 3-4 months.

Detection of anthrax-specific antibody levels. Anthrax specific antibody levels were measured by ELISA as described previously.¹² High binding ELISA plates were coated with 100 ng per well of anthrax protective antigen (List Biological Laboratories Inc., Campbell, Calif., USA) in carbonate buffer, pH 9.6 and incubated at 37° C. for 1 hour, then overnight at 4° C. The plates were washed 3-5 times with phosphate buffered saline containing 0.5% Tween 20 (PBST). This washing step was repeated between each of the following steps. Next, the plates were blocked for 1 hour at 37° C. with 100 μl per well of 1% (w/v) bovine serum albumin in PBST (1% BSA). Serial dilutions of all serum samples were prepared in 1% BSA, loaded 100 μl per well and incubated for 1 hour at 37° C. The secondary antibody, Anti-Mouse IgG (H+L) (SeraCare, Milford, Mass., USA) or Anti-Deer IgG (H+L) (SeraCare, Milford, Mass., USA) was diluted, 1:5000 or 1:500 respectively, in 1% BSA and loaded 100 μl to a well with a 1 hour incubation at 37° C. TMB/E Substrate (Sigma-Aldrich, St. Louis, Mo., USA) was added to each well and the reaction was stopped after 12 minutes with the addition of 100 μl of 0.5 M H₂SO₄. The optical density of all wells was read on a Tecan Infinite F50 Plate Reader at 450 nm. Samples (n=5) from each time point, at each dilution were run in duplicate and are reported as average absorbance values for a single dilution for all vaccination groups at each time point. Also reported are the measured antibody titers in mouse serum as the reciprocal of the maximum dilution giving an absorbance greater than two standard deviations above the unvaccinated control.

Table 2. Serum antibody titers were determined by end-point dilution ELISA from mice vaccinated subcutaneously and orally with Empty Capsules, the Sterne Vaccine, Low Dose Capsules or High Dose Capsules. BALBc/J mice were either subcutaneously injected or orally inoculated with 10⁶ unencapsulated B. anthracis Sterne strain 34F2 spores or 10⁶ encapsulated Sterne spores in Low Dose Capsules. An additional group of mice were subcutaneously injected with 10⁹ encapsulated Sterne spores in High Dose Capsules. Control groups received empty capsules. Serum samples were collected at 0, 15, 31, 43- and 55-days post vaccination and the antibody titer was analyzed by end-point dilution ELISA. The resulting antibody titers are reported as the reciprocal of the maximum dilution giving an absorbance greater than two standard deviations above the unvaccinated control.

TABLE 2
Serum antibody titers as determined by end-point dilution
ELISA from mice vaccinated subcutaneously
and orally with Empty Capsules, the Sterne
Vaccine, Low Dose Capsules or High Dose Capsules.
Anti-Anthrax Protective Antigen Antibody Titers

| | Vaccine | Day 0 | Day 15 | Day 31 | Day 43 | Day 55 |
|---|---|---|---|---|---|---|
| SC | Empty Capsules | ND | ND | ND | ND | ND |
| | Sterne Vaccine | ND | 8,000 | 16,000 | 32,000 | 32,000 |
| | Low Dose | ND | 8,000 | 32,000 | 64,000 | 32,000 |
| | Capsules | | | | | |
| | High Dose | ND | 32,000 | 128,000+ | 128,000+ | 128,000+ |
| | Capsules | | | | | |
| Oral | Empty Capsules | ND | ND | ND | ND | ND |
| | Sterne Vaccine | ND | ND | ND | ND | ND |
| | Low Dose | ND | 125 | 500 | 1,000 | 1,000+ |
| | Capsules | | | | | |

Values reported are reciprocal dilutions.
+represents samples that had not yet dropped below 50% protection at the highest dilution made.
ND = Not detectable.

Lethal toxin neutralization assays. Toxin neutralization assays were performed to determine the ability of collected serum samples to inhibit the cytotoxicity of anthrax lethal toxin (LeTx) in vitro.^{14,15,48} J774A.1 macrophages were cultured in Dulbecco's modified eagle medium (DMEM, HyClone) with 10% (w/v) fetal bovine serum (FBS) and 1% (w/v) penicillin. Upon reaching confluency, the cells were harvested and quantified using a hemocytometer, then brought to a final concentration of 5×10⁴ cells/ml. Cells were added to a 96-well flat-bottom tissue culture plate at 200 μl/well and incubated overnight at 37° C. in 5% CO₂. LeTx was prepared by adding lethal factor (List Biological Laboratories Inc., Campbell, Calif., USA) and protective antigen (List Biological Laboratories Inc., Campbell, Calif., USA) to DMEM containing 10% FBS and no antibiotic at concentrations of 0.25 μg/ml and 0.1 μg/ml, respectively. The LeTx mixture was used to make serial dilutions of the collected mouse or deer serum samples from each time point on a separate 96-well cell culture plate and then incubated for 1 hour at 37° C., 5% CO₂. The media was removed from the prepared macrophage plate and replaced with 100 μl/well of the serum LeTx mixture in triplicate. After incubating for 4 hours at 37° C., 5% CO₂, 10 μl of MTT (3-[4,5-dimethylthiazol-2-yl]-2,5-diphenyltetrazolium bromide; Roche, Basel, Switzerland) was added to each well and incubated for another 4 hours at 37° C., 5% CO₂. Any remaining metabolically active cells reduced MTT, a yellow tetrazolium salt, to purple formazan crystals using NAD(P)H-dependent oxidoreductase enzymes. The insoluble formazan crystals were dissolved by adding 100 μl of solubilization solution (Roche, Basel, Switzerland) to each well and plates were incubated overnight at 37° C., 5% CO₂. The optical density of each well was read at 595 nm using a Tecan Infinite F50 Plate Reader. Cells that were exposed to only LeTx and no serum were used as a positive control. Cells that did not receive any LeTx or serum were used to determine 100% cell viability. Cells that did not receive any LeTx or serum were used to determine 100% cell viability. The LeTx neutralizing abilities of collected serum samples are reported as average absorbance values for a single dilution for all vaccination groups at each time point from all repetitions of the experiment. Also included are the LeTx neutralizing antibody titers (NT50) reported as the maximum dilution that resulted in over 50% protection which were calculated as

$$NT 50 = \frac{( mean sample - mean LeTx control )}{( mean media control - mean LeTx control )} \times 100.$$

Statistical analysis. Differences between starting and ending titers for Sterne spore responses to GI fluids, and the difference between microcapsule image pixel intensities were determined by two-sided Student's t-tests with p-values<0.05 considered significant. Across all other experiments, results are expressed as mean values±standard deviations for all replicates at each time point for each group. Statistical analysis was performed using one-way ANOVA followed by the Tukey-Kramer HSD test with p-values<0.05 considered significant.

Table 3. Neutralizing antibody titers against anthrax lethal toxin were determined by toxin neutralization assays with serial serum dilutions from mice vaccinated subcutaneously and orally with Empty Capsules, the Sterne Vaccine, Low Dose Capsules or High Dose Capsules. Serum was collected from mice at 0, 15, 31, 43- and 55-days post subcutaneous or oral vaccination with 10⁶ unencapsulated B. anthracis Sterne strain 34F2 spores, 10⁶ encapsulated Sterne spores in Low Dose Capsules or 10⁹ encapsulated Sterne spores in High Dose Capsules. Control groups received Empty Capsules. Diluted serum samples were pre-incubated with LeTx then added to J774A.1 cells and resulting cell viability was assessed with MTT dye. The LeTx neutralizing antibody titers are reported as the reciprocal of the maximum dilution that resulted in over 50% protection which were calculated as:

$$NT 50 = \frac{( mean sample - mean LeTx control )}{( mean media control - mean LeTx control )} \times 100.$$

TABLE 3
Neutralizing antibody titers against anthrax lethal toxin as determined
by toxin neutralization assays with serial serum dilutions from mice
vaccinated subcutaneously and orally with Empty Capsules,
the Sterne Vaccine, Low Dose Capsules or High Dose Capsules.
Anthrax Lethal Toxin Neutralizing Antibody Titers

| | Vaccine | Day 0 | Day 15 | Day 31 | Day 43 | Day 55 |
|---|---|---|---|---|---|---|
| SC | Empty Capsules | ND | ND | ND | ND | ND |
| | Sterne Vaccine | ND | 200+ | 50 | 100+ | 100+ |
| | Low Dose Capsules | ND | 200+ | 200+ | 200+ | 200+ |
| | High Dose Capsules | ND | 800+ | 800+ | 800+ | 800+ |
| | Empty Capsules | ND | ND | ND | ND | ND |
| | Oral Sterne Vaccine | ND | ND | ND | ND | ND |
| | Low Dose Capsules | ND | 100+ | 100 | 50 | 100+ |

Values reported are reciprocal dilutions.
+represents samples that had not yet dropped below 50% protection at the highest dilution made.
ND = Not detectable.

In one embodiment, the present invention includes an oral immunization against Bacillus anthracis comprising, consisting essentially of, or consisting of: B. anthracis Sterne strain 34F2 spores suspended in alginate and coated with a shell containing poly-L-lysine (PLL), vitelline protein B (VpB), or both in an amount sufficient to protect an animal or human from a lethal dose of anthrax. In one aspect, the composition further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores. In another aspect, the vitelline protein B is encoded by Fasciola hepatica. In another aspect, B. anthracis Sterne strain 34F2 spores encapsulated in alginate and coated with the VpB shell survive exposure to gastric juices. In another aspect, the immunization further comprises a pharmaceutically acceptable carrier, a bait additive, or both. In another aspect, the oral immunization further includes an outer alginate shell surrounding the protein shell that comprises an alginate bead or an alginate microsphere. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM. In another aspect, the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

In another embodiment, the present invention includes a vaccine comprising, consisting essentially of, or consisting of: B. anthracis Sterne strain 34F2 spores suspended in alginate and coated with a shell containing poly-L-lysine (PLL), a vitelline protein B (VpB), or both, wherein the spores are provided in an amount sufficient to protect an animal or human from a lethal dose of anthrax formulated for oral administration. In one aspect, the composition further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores. In another aspect, the vitelline protein B is a recombinant protein. In another aspect, the vitelline protein B is encoded by Fasciola hepatica. In another aspect, B. anthracis Sterne strain 34F2 spores encapsulated in alginate and coated with the VpB shell survive exposure to gastric condition. In another aspect, the immunization further comprises a pharmaceutically acceptable carrier, a bait additive, or both. In another aspect, the vaccine further comprises an outer shell surrounding the protein shell comprising an alginate bead or an alginate microsphere. In another aspect, the alginate further comprises an amount of D-alanine sufficient to prevent germination of the B. anthracis Sterne strain 34F2 spores In another aspect, the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM. In another aspect, the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

In one embodiment, the present invention includes a method for prophylaxis, amelioration of symptoms, or any combinations thereof against Bacillus anthracis in a human or animal subject comprising, consisting essentially of, or consisting of, the steps of: identifying the human or animal subject in need of the prophylaxis, amelioration of symptoms, or any combinations thereof against Bacillus anthracis; and administering a therapeutically effective amount of an attenuated oral immunization against Bacillus anthracis comprising: B. anthracis Sterne strain 34F2 spores suspended in alginate, and the alginate is coated with a shell containing poly-L-lysine (PLL), a vitelline protein B (VpB), or both, wherein the immunization is provided in an amount sufficient to protect an animal or human from a lethal dose of anthrax. In one aspect, the composition further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores. In another aspect, the vitelline protein B is a recombinant protein. In another aspect, the vitelline protein B is encoded by Fasciola hepatica. In another aspect, the B. anthracis Sterne strain 34F2 spores encapsulated in alginate and coated with the VpB shell survive exposure to gastric juices. In another aspect, the immunization further comprises a pharmaceutically acceptable carrier, a bait additive, or both. In another aspect, the method further comprises an outer shell surrounding the protein shell comprising an alginate bead or an alginate microsphere. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM. In another aspect, the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

In one embodiment, the present invention includes a method of making an attenuated oral vaccine against anthrax (Bacillus anthracis) comprising, consisting essentially of, or consisting of: suspending a B. anthracis Sterne strain 34F2 spores in alginate, and coating the alginate with a protein shell comprising: poly-L-lysine (PLL), vitelline protein B (VpB), or both, wherein the protein shell protects the spores from exposure to gastric conditions, wherein the amount of the vaccine is sufficient to protect an animal or human from a lethal dose of anthrax. In one aspect, the composition further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores. In another aspect, the vitelline protein B is a recombinant protein. In another aspect, the vitelline protein B is from Fasciola hepatica. In another aspect, the immunization further comprises a pharmaceutically acceptable carrier, a bait, or both. In another aspect, the method, further comprises encapsulating the spores in an alginate bead or an alginate microsphere. In another aspect, the alginate further comprises an amount of D-alanine sufficient to prevent germination of the B. anthracis Sterne strain 34F2 spores. In another aspect, the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM. In another aspect, the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Hugh-Jones, M. E. & de Vos, V. Anthrax and wildlife. Rev. Sci. Tech. 21, 359-83 (2002).
2. Bacillus anthracis and Anthrax. Bacillus anthracis and Anthrax (Wiley-Blackwell, 2011). doi: 10.1002/9780470891193
3. Carlson, C. J. et al. The global distribution of Bacillus anthracis and associated anthrax risk to humans, livestock and wildlife. Nat. Microbiol. 4, 1337-1343 (2019).
4. Sterne, M. Distribution and economic importance of anthrax. Fed Proc 26, 1493-1495 (1967).
5. Kracalik, I. et al. Changing livestock vaccination policy alters the epidemiology of human anthrax, Georgia, 2000-2013. Vaccine 35, 6283-6289 (2017).
6. Scorpio, A., Blank, T E., Day, W. A. & Chabot, D. J. Anthrax vaccines: Pasteur to the present. Cellular and Molecular Life Sciences 63, 2237-2248 (2006).
7. Anthrax in Humans and Animals. in (ed. Turnbull, P.) (World Health Organization and International Office of Epizootics, 2008).
8. Brossier, F., Mock, M. & Sirard, J. C. Antigen delivery by attenuated Bacillus anthracis: New prospects in veterinary vaccines. J. Appl. Microbiol. 87, 298-302 (1999).
9. Hugh-Jones, M. & Blackburn, J. The ecology of Bacillus anthracis. Mol. Aspects Med. 30, 356-367 (2009).
10. Fasanella, A., Galante, D., Garofolo, G. & Jones, M. H. Anthrax undervalued zoonosis. Vet Microbiol 140, 318-331 (2010).
11. Hudson, M. J. et al. Bacillus anthracis: Balancing innocent research with dual-use potential. International Journal of Medical Microbiology 298, 345-364 (2008).
12. Benn Felix, J., Chaki, S. P., Ficht, T. A., Rice-Ficht, A. C. & Cook, W. Bacillus anthracis Sterne Strain 34F2 Vaccine Antibody Dose Response by Subcutaneous and Oral Administration. Poult Fish Wildl Sci 7, 206 (2019).
13. Aloni-Grinstein, R. et al. Oral Spore Vaccine Based on Live Attenuated Nontoxinogenic Bacillus anthracis Expressing Recombinant Mutant Protective Antigen. Infect. Immun 73, 4043-4053 (2005).
14. Gorantala, J. et al. A plant based protective antigen [PA(dIV)] vaccine expressed in chloroplasts demonstrates protective immunity in mice against anthrax. Vaccine 29, 4521-4533 (2011).
15. Gorantala, J. et al. Generation of protective immune response against anthrax by oral immunization with protective antigen plant-based vaccine. J. Biotechnol. 176, 1-10 (2014).
16. Sim, B. K. L. et al. Protection against inhalation anthrax by immunization with Salmonella enterica serovar Typhi Ty21a stably producing protective antigen of Bacillus anthracis. npj Vaccines (2017). doi:10.1038/s41541-017-0018-4
17. Brossier, F., Levy, M. & Mock, M. Anthrax spores make an essential contribution to vaccine efficacy. Infect. Immun 70, 661-664 (2002).
18. Choo, M.-K. et al. TLR sensing of bacterial spore-associated RNA triggers host immune responses with detrimental effects. J. Exp. Med. 214, 1297-1311 (2017).
19. Rice-Ficht, A. C., Arenas-Gamboa, A. M., Kahl-McDonagh, M. M. & Ficht, T. A. Polymeric particles in vaccine delivery. Current Opinion in Microbiology 13, 106-112 (2010).
20. Cook, M. T., Tzortzis, G., Charalampopoulos, D. & Khutoryanskiy, V. V. Microencapsulation of probiotics for gastrointestinal delivery. J. Control. Release 162, 56-67 (2012).
21. Simó, G., Fernández□Fernández, E., Vila□Crespo, J., Ruipérez, V. & Rodríguez□Nogales, J. M. Research progress in coating techniques of alginate gel polymer for cell encapsulation. Carbohydrate Polymers (2017). doi:10.1016/j.carbpol.2017.04.013
22. Gombotz, W. R. & Wee, S. F. Protein release from alginate matrices. Advanced Drug Delivery Reviews 64, 194-205 (2012).
23. Sundar, S., Kundu, J. & Kundu, S. C. Biopolymeric nanoparticles. Sci. Technol. Adv. Mater. 11, 014104 (2010).
24. De, S. & Robinson, D. Polymer relationships during preparation of chitosan-alginate and poly-1-lysine-alginate nanospheres. J. Control. Release 89, 101-112 (2003).
25. Martín, M. J., Lara-Villoslada, F., Ruiz, M. A. & Morales, M. E. Microencapsulation of bacteria: A review of different technologies and their impact on the probiotic effects. Innov. Food Sci. Emerg. Technol. 27, 15-25 (2015).
26. Thu, B. et al. Alginate polycation microcapsules: I. Interaction between alginate and polycation. Biomaterials 17, 1031-1040 (1996).
27. Herbert Waite, J. & Rice-Ficht, A. C. Eggshell precursor proteins of Fasciola hepatica, II. Microheterogeneity in vitelline protein B. Mol. Biochem. Parasitol. 54, 143-151 (1992).
28. Rice-Ficht, A. C., Dusek, K. A., John Kochevar, G. & Herbert Waite, J. Eggshell precursor proteins of Fasciola hepatica, I. Structure and expression of vitelline protein B. Mol. Biochem. Parasitol. 54, 129-141 (1992).
29. Arenas-Gamboa, A. M. et al. Oral vaccination with microencapsuled Strain 19 vaccine confers enhanced protection against Brucella abortus strain 2308 challenge in Red deer (Cervus elaphus elaphus). J. Wildl. Dis. 45, 1021-1029 (2009).
30. Arenas-Gamboa, A. M., Ficht, T. A., Kahl-McDonagh, M. M., Gomez, G. & Rice-Ficht, A. C. The Brucella abortus S19 ΔvjbR live vaccine candidate is safer than S19 and confers protection against wild-type challenge in BALB/c mice when delivered in a sustained-release vehicle. Infect. Immun 77, 877-884 (2009).
31. Arenas-Gamboa, A. M., Ficht, T. A., Kahl-McDonagh, M. M. & Rice-Ficht, A. C. Immunization with a single dose of a microencapsulated Brucella melitensis mutant enhances protection against wild-type challenge. Infect. Immun 76, 2448-2455 (2008).
32. Arenas-Gamboa, A. M. et al. Enhanced Immune Response of Red Deer (Cervus elaphus) to Live RB51 Vaccine Strain Using Composite Microspheres. 45, 165-173 (2009).
33. Cook, M. T., Tzortzis, G., Charalampopoulos, D. & Khutoryanskiy, V. V. Production and evaluation of dry alginate-chitosan microcapsules as an enteric delivery vehicle for probiotic bacteria. Biomacromolecules 12, 2834-2840 (2011).
34. Meeusen, E. N. T., Walker, J., Peters, A., Pastoret, P. P. & Jungersen, G. Current status of veterinary vaccines. Clin. Microbiol. Rev. 20, 489-510 (2007).
35. CDC & Ncird. U.S. Vaccines: Table 1 and Table 2.
36. Vela Ramirez, J. E., Sharpe, L. A. & Peppas, N. A. Current state and challenges in developing oral vaccines. Advanced Drug Delivery Reviews (2017). doi:10.1016/j.addr.2017.04.008
37. Gåserød, O., Sannes, A. & Skjåk-Bræk, G. Microcapsules of alginate-chitosan. II. A study of capsule stability and permeability. Biomaterials 20, 773-783 (1999).
38. Thu, B. et al. Alginate polycation microcapsules: II. Some functional properties. Biomaterials 17, 1069-1079 (1996).
39. Kuo, C. K. & Ma, P. X Maintaining dimensions and mechanical properties of ionically crosslinked alginate hydrogel scaffolds in vitro. J. Biomed. Mater. Res. —Part A (2008). doi:10.1002/jbm.a.31375
40. Chuang, J. J. et al. Effects of pH on the Shape of Alginate Particles and Its Release Behavior. Int. J. Polym. Sci. 2017, (2017).
41. Rasel, M. A. T. & Hasan, M. Formulation and evaluation of floating alginate beads of diclofenac sodium. Dhaka Univ. J. Pharm. Sci. (2012). doi:10.3329/dujps.v11i1.12484
42. Iyer, C. & Kailasapathy, K. Effect of Co-encapsulation of Probiotics with Prebiotics on Increasing the Viability of Encapsulated Bacteria under In Vitro Acidic and Bile Salt Conditions and in Yogurt. J. Food Sci. 70, M18-M23 (2005).
43. Li, X. Y. et al. Preparation of alginate coated chitosan microparticles for vaccine delivery. BMC Biotechnol. 8, 1-11 (2008).
44. Praepanitchai, O. A., Noomhorm, A., Anal, A. K. & Potes, M. E. Survival and Behavior of Encapsulated Probiotics (Lactobacillus plantarum) in Calcium-Alginate-Soy Protein Isolate-Based Hydrogel Beads in Different Processing Conditions (pH and Temperature) and in Pasteurized Mango Juice. Biomed Res. Int. 2019, (2019).
45. Storni, T., Kiindig, T. M., Senti, G. & Johansen, P Immunity in response to particulate antigen-delivery systems. Advanced Drug Delivery Reviews (2005). doi:10.1016/j.addr.2004.09.008
46. Csaba, N. et al. Trimethyl chitosan nanoparticles encapsulated protective antigen Protects the mice against anthrax. Front. Immunol. (2018). doi:10.3389/fimmu.2018.00562
47. Shakya, K. P., Hugh-Jones, M. E. & Elzer, P. H. Evaluation of immune response to orally administered Sterne strain 34F2 anthrax vaccine. Vaccine 25, 5374-5377 (2007).
48. Hanson, J. F., Taft, S. C. & Weiss, A. A. Neutralizing antibodies and persistence of immunity following anthrax vaccination. Clin. Vaccine Immunol. 13, 208-213 (2006).
49. Reuveny, S. et al. Search for correlates of protective immunity conferred by anthrax vaccine. Infect. Immun 69, 2888-2893 (2001).
50. Welkos, S. L., Keener, T. J. & Gibbs, P. H. Differences in Susceptibility of Inbred Mice to Bacillus-anthracis. Infect. Immun 51, 795-800 (1986).
51. Jang, S.-F. et al. Size discrimination in rat and mouse gastric emptying. Biopharm. Drug Dispos. 34, 107-124 (2013).
52. McGhee, J. R. et al. The mucosal immune system: from fundamental concepts to vaccine development. Vaccine 10, 75-88 (1992).
53. Vandamme, T. F. & Ellis, K. J. Issues and challenges in developing ruminal drug delivery systems. Adv. Drug Deliv. Rev. 56, 1415-1436 (2004).
54. Xu, Y., Liang, X., Chen, Y., Koehler, T. M. & Hook, M. Identification and biochemical characterization of two novel collagen binding MSCRAMMs of Bacillus anthracis. J Biol Chem 279, 51760-51768 (2004).
55. Russell, B. H., Vasan, R., Keene, D. R. & Xu, Y. Bacillus anthracis internalization by human fibroblasts and epithelial cells. Cell. Microbiol. 9, 1262-1274 (2007).
56. Russell, B. H., Vasan, R., Keene, D. R., Koehler, T. M. & Xu, Y. Potential dissemination of Bacillus anthracis utilizing human lung epithelial cells. Cell. Microbiol. 10, 945-957 (2008).
57. Jenkins, S. A. & Xu, Y. Characterization of Bacillus anthracis Persistence In Vivo. PLoS One 8, 1-9 (2013).
58. Basu, S. et al. Role of Bacillus anthracis spore structures in macrophage cytokine responses. Infect. Immun 75, 2351-2358 (2007).
59. Parish, J., Rivera, J. & Boland, H. Understanding the ruminant animal digestive system. 1-5 (2009).

1. An oral immunization against Bacillus anthracis comprising:
B. anthracis Sterne strain 34F2 spores suspended in alginate and coated with a shell containing poly-L-lysine (PLL), a vitelline protein B (VpB), or both in an amount sufficient to protect an animal or human from a lethal dose of anthrax.

2. The oral immunization of claim 1, wherein the oral immunization further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores; further comprises an outer shell surrounding the protein shell that comprises an alginate bead or an alginate microsphere; or a pharmaceutically acceptable carrier, a bait, or both.

3. The oral immunization of claim 1, wherein the vitelline protein B is a recombinant protein.

4. The oral immunization of claim 1, wherein the vitelline protein B is encoded by Fasciola hepatica.

5. The oral immunization of claim 1, wherein the B. anthracis Sterne strain 34F2 spores survive exposure to gastric conditions.

6. (canceled)

7. (canceled)

8. The oral immunization of claim 1, wherein at least one of:
the alginate further comprises an amount of D-alanine sufficient to prevent germination of the B. anthracis Sterne strain 34F2 spores;
the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM; or
the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

9. (canceled)

10. (canceled)

11. A vaccine comprising:
B. anthracis Sterne strain 34F2 spores suspended in alginate and coated with a shell containing poly-L-lysine (PLL), a vitelline protein B (VpB), or both, wherein the spores are provided in an amount sufficient to protect an animal or human from a lethal dose of anthrax formulated for oral administration.

12. The vaccine of claim 11, wherein the oral immunization further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores; further comprises an outer shell surrounding the protein shell that comprises an alginate bead or an alginate microsphere; or a pharmaceutically acceptable carrier, a bait, or both.

13. The vaccine of claim 11, wherein the vitelline protein B is a recombinant protein.

14. The vaccine of claim 11, wherein the vitelline protein B is encoded by Fasciola hepatica.

15. The vaccine of claim 11, wherein the B. anthracis Sterne strain 34F2 spores survive exposure to gastric conditions.

16. (canceled)

17. (canceled)

18. The vaccine of claim 11, wherein at least one of:
the alginate further comprises an amount of D-alanine sufficient to prevent germination of the B. anthracis Sterne strain 34F2 spores;
the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM; or
the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

19. (canceled)

20. (canceled)

21. A method for prophylaxis, amelioration of symptoms, or any combinations thereof against Bacillus anthracis in a human or animal subject comprising the steps of:
identifying the human or animal subject in need of the prophylaxis, amelioration of symptoms, or any combinations thereof against Bacillus anthracis; and
administering a therapeutically effective amount of an attenuated oral immunization against Bacillus anthracis comprising:
B. anthracis Sterne strain 34F2 spores suspended in an alginate, and the alginate is coated with a shell containing poly-L-lysine (PLL), a vitelline protein B (VpB), or both, wherein the immunization is provided in an amount sufficient to protect an animal or human from a lethal dose of anthrax.

22. The method of claim 21, wherein the oral immunization further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores; further comprises an outer shell surrounding the protein shell that comprises an alginate bead or an alginate microsphere; or a pharmaceutically acceptable carrier, a bait, or both.

23. The method of claim 21, wherein the vitelline protein B is a recombinant protein.

24. The method of claim 21, wherein the vitelline protein B is encoded by Fasciola hepatica.

25. The method of claim 21, wherein the B. anthracis Sterne strain 34F2 spores survive exposure to gastric conditions.

26. (canceled)

27. (canceled)

28. The method of claim 21, wherein at least one of:
the alginate further comprises an amount of D-alanine sufficient to prevent germination of the B. anthracis Sterne strain 34F2 spores;
the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM; or
the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

29. (canceled)

30. (canceled)

31. A method of making an attenuated oral vaccine against anthrax (Bacillus anthracis) comprising:
suspending a B. anthracis Sterne strain 34F2 spores in alginate, and
coating the alginate with a protein shell comprising: poly-L-lysine (PLL), vitelline protein B (VpB), or both, and an external coating of alginate wherein the protein shell protects the spores from exposure to gastric juices, wherein an external alginate coating neutralizes positively charged amino acids and the VpB causes sustained release, wherein the amount of the vaccine is sufficient to protect an animal or human from a lethal dose of anthrax.

32. The method of claim 0, wherein the oral immunization further comprises at least one of: an adjuvant, a delivery vehicle for at least one of a B. anthracis protective antigen, a B. anthracis edema factor, or a B. anthracis lethal factor, which are encapsulated separately, together, or in combination with B. anthracis Sterne spores; further comprises an outer shell surrounding the protein shell that comprises an alginate bead or an alginate microsphere; or a pharmaceutically acceptable carrier, a bait, or both.

33. The method of claim 0, wherein the vitelline protein B is a recombinant protein.

34. The method of claim 0, wherein the vitelline protein B is from Fasciola hepatica.

35. (canceled)

36. (canceled)

37. The method of claim 0, wherein at least one of:
the alginate further comprises an amount of D-alanine sufficient to prevent germination of the B. anthracis Sterne strain 34F2 spores;
the alginate further comprises an amount of D-alanine at an amount of 0.1, 0.2, 0.3, 0.4, 0.5. 0.75, 1, 2, 2.5, 3, 4, 5, 6, 7, 8, 9, 10 or more mM; or
the alginate is at 0.1, 0.2, 0.3, 0.4, 0.5. 0.75, 1, 1.25, 1.5, 1.75, 2, 2.25, 2.5, 2.75, 3, 4, 5, 6, 7, 8, 9, 10% weigh to volume (w/v).

38. (canceled)

39. (canceled)
